(19) United States
(12) Patent Application Publication
(10) Pub. No.: US 2023/0194537 A1
(43) Pub. Date: Jun. 22, 2023

(54) COMPOUNDS FOR THE DETECTION OF GLYCANS

(71) Applicant: LIFE TECHNOLOGIES CORPORATION, Carlsbad, CA (US)

(72) Inventors: Shaheer H. KHAN, Foster City, CA (US); Jonathan BONES, Dublin (IE); Csaba VARADI, Miskolc (HU)

(21) Appl. No.: 16/494,535

(22) PCT Filed: Mar. 16, 2018
(86) PCT No.: PCT/US2018/022988

§ 371 Date: Dec. 12, 2019

Related U.S. Application Data
(60) Provisional application No. 62/472,230, filed on Mar. 16, 2017.

(51) Int. Cl. G01N 33/58; G01N 33/68

(52) U.S. Cl. CPC G01N 33/582; G01N 33/68; G01N 2400/12

(57) ABSTRACT

The invention relates to the analysis of carbohydrates, such as N-glycans and O-glycans found on proteins. The invention relates, in part, to glycan labeling with formulas/compounds enhancing their detection and/or analysis by methods such as capillary electrophoresis, liquid chromatography and mass spectrometry. These detection methods may be useful in studying glycosylation patterns of biological or medical samples, or for assessing protein production, protein quality/purity, for comparing innovator and biosimilar glycosylated proteins, or for selecting proteins with the desired glycosylation.

### FIELD OF THE INVENTION

This invention relates to the field of glycan analysis, for example, in glycoproteins, and further, to the analysis of glycans by fluorescence and mass spectrometry techniques.

### BACKGROUND OF THE INVENTION

Proteins produced in cells may be modified after translation by the addition of covalently-linked, linear or branched chains of carbohydrates. These protein-carbohydrate conjugates are referred to as glycoproteins; the point at which the carbohydrate is attached is referred to as a glycosylation site. Attached polysaccharides or oligosaccharides are referred to as glycans. A wide range of glycans are found on the different glycosylation sites of particular glycoproteins. The particular pattern of glycans on a particular glycoprotein is determined by the specific cell line that produces the glycoprotein and the conditions under which the cells are grown.

Since glycans conjugated to a protein can affect characteristics critical to its function, including pharmacokinetics, stability, bioactivity, or immunogenicity, it is important in many uses to determine which glycans are present. Thus, as glycans are released from a glycoprotein during analysis, it is useful to label them. N-glycans are typically labeled at their free-reducing terminus and are analyzed by methods such as high performance liquid chromatography (HPLC), capillary electrophoresis (CE), carbohydrate gel electrophoresis, fluorescence analysis, mass spectrometry (MS) and others. U.S. Pat. Nos. 8,124,792 and 8.445,292 describe some methods of glycan analysis, and are hereby incorporated by reference.

There is a need in the art for additional labels and methods that provide rapid labeling of N-glycans under mild conditions without causing the degradation of labile components, and that can provide high sensitivity of detection by both fluorescence and by MS or by HPLC/CE).

### SUMMARY OF THE INVENTION

The present disclosure provides compounds comprising (a) at least one fluorescent group such as an aromatic or substituted aromatic ring; (b) at least one group capable of binding to an aldehyde or ketone present on an analyte of interest, for e.g.; to a glycoprotein; and (c) at least one negatively charged group that can be detected by negative ion mass spectrometry. These compounds can be used to label and detect the glycans. Therefore, the present disclosure provides methods for labeling or binding aldehyde, ketone-, glycosylamine-containing molecules or analytes, for e.g., glycans.

The present disclosure also provides methods for comparative quantitation of glycans using the ¹²C/¹³C labeled isotopologues of the compounds described herein (including for e.g., sulfanilic acid, its derivatives or salts thereof), thereby providing indicating structural characterization and quantitation of glycan alterations.

Accordingly, the present disclosure provides fluorescent, MS active compounds that comprise three functional components: (a) a molecular scaffold comprising an aromatic group (for fluorescence), including but not limited to benzene, (N- or S-) substituted benzene, naphthalene, (N- or S-) substituted naphthalene, anthracene, (N- or S-) substituted anthracene, pyrene, (N- or S-) substituted pyrene, derivatives or salts thereof of any of the preceding compounds; (b) a group for coupling to glycan aldehydes, or glycan ketones, or a glycosylamine, for e.g., an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds; and (c) a negatively charged group designed to enhance detection in negative ion mass spectrometry (MS), including but not limited to, sulfonic acid and their derivatives or salts thereof, phosphoric acid and their derivatives or salts thereof, carboxylic acid and their derivatives or salts thereof.

The present disclosure provides methods for detecting the resultant labeled glycans that may be highly fluorescent, thereby providing an excellent method for detection via fluorescence; and that may be highly charged thereby enhancing the detection in negative ion mass spectrometry (MS). The labeled glycans can also be analyzed using capillary electrophoresis (CE), liquid chromatography (LC, UPLC, UHPLC, etc.) or similar techniques. The present disclosure provides methods for combining the above techniques (twoplexed/duplexed/multiplexed) for analysis, for e.g., UPLC-fluorescence—MS analysis, CE-LC, LC-MS, CE-MS, MS-MS, etc. These techniques or their combinations can also be set up for automation.

Certain embodiments provide a method of labeling one or more glycans in a glycoprotein sample, the method comprising:
(a) deglycosylating the one or more glycans from the glycoprotein;
(b) contacting the one or more deglycosylated glycans of step (a) with a fluorescent reagent of Formula I:

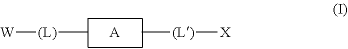

wherein,
A is an aromatic component;
X is a molecular scaffold substituent designed to react with a glycan aldehyde, ketone or an aminoglycan;
optionally attached L and L′ are linkers, that are independently (CH₂)ₙ, (OCH₂CH₂)ₘ,
(CH₂)ₙ(OCH₂CH₂)ₚ or (OCH₂CH₂)ₚ(CH₂)ₙ; where n is 0 to 8, and m and p are independently 0 to 4; and,
W is a negatively charged substituent for detection;
(c) reacting the one or more deglycosylated glycans and the fluorescent reagent to form a fluorescent target product.

In certain embodiments, the fluorescent target product is an N-labeled or an 0-labeled glycan.

In certain embodiments,

A is benzene, (N- or S-) substituted benzene, naphthalene, (N- or S-) substituted naphthalene, anthracene, (N- or S-) substituted anthracene, pyrene, (N- or S-) substituted pyrene, or derivatives or salts thereof;

X is an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives or salts thereof; and,

W is sulfonic acid and their derivatives or salts thereof, phosphoric acid and their derivatives or salts thereof, carboxylic acid and their derivatives or salts thereof.

The activated carboxylic group is selected from: a succinimidyl, N-hydroxysuccinimidyl (NHS) ester, N-hydroxysuccinimidyl (NHS) cyanate, N-hydroxysuccinimidyl (NHS) thiocyanate, N-hydroxysuccinimidyl (NHS) isothiocyanate, and N-hydroxysuccinimidyl (NHS) isocyanate or a salt thereof.

In certain embodiments, the fluorescent reagent of Formula I is a benzenesulfonic acid derivative or a salt thereof.

In certain embodiments, fluorescent reagent is a compound of Formula II, III or IV, its derivatives or salt thereof:

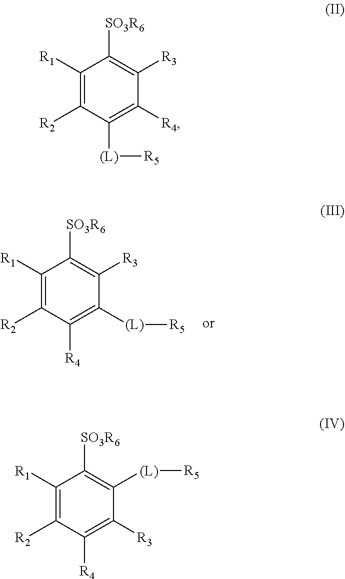

wherein,
R₁, R₂, R₃ and R₄ are independently, H, C₁-C₆-alkyl, C₁-C₆ alkoxy or halogen;
optionally attached linker L is (CH₂)ₙ, (OCH₂CH₂)ₘ, (CH₂CH₂O)ₘ, (CH₂)—(OCH₂CH₂)ₚ or (OCH₂CH₂)ₚ(CH₂)ₙ; where n is 0 to 8, and m and p are independently 0 to 4;
R₅ is a primary amino group, hydrazine or substituted hydrazine, hydrazide or substituted hydrazide, hydrazone or substituted hydrazone, oxime or substituted oxime, sulfonyl chloride, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds;
R₆ is H or O⁻ or OM where M is a metal ion or an ammonium ion.

In certain embodiments, R₁, R₂, R₃ and R₄ are independently H, methyl, ethyl, C₃-C₆ alkyl, C₁-C₆ alkoxy or halogen.

In certain embodiments, the fluorescent reagent is a compound of Formula V, VI or VII, its derivatives or salt thereof:

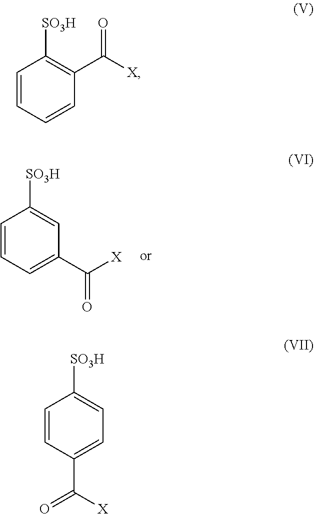

wherein, X is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof.

In certain embodiments, the fluorescent reagent is a compound of Formula IX, X or XI, its derivatives or salt thereof:

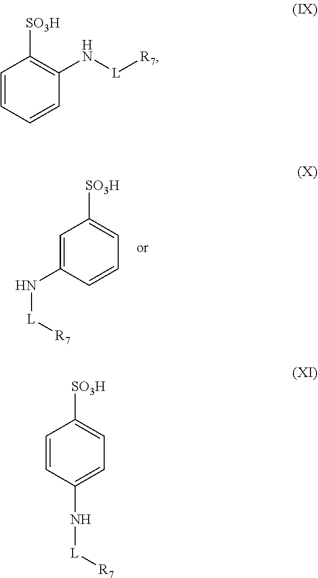

wherein,
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ where n is 0 to 8 and m and p are independently 0 to 4;
if n, m or p is 0, R₇ is H; and,
if n is 1 to 8, or if m or p are independently 1 to 4, then,
R₇ is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds.

In certain embodiments, the fluorescent reagent is any of Compounds 1, 2 or 3, their derivatives or salts thereof:

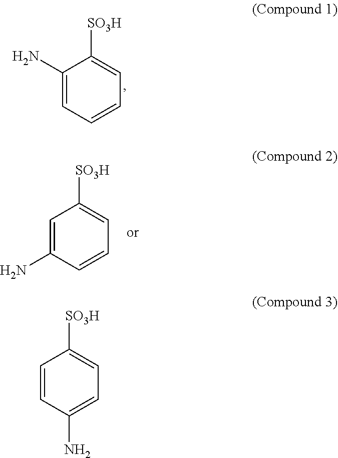 [SMILES: Nc1ccccc1S(=O)(=O)O ; Nc1cccc(S(=O)(=O)O)c1 ; Nc1ccc(S(=O)(=O)O)cc1]

In certain embodiments, the fluorescent reagent is Compound 9 or Compound 10, their derivatives or salts thereof:

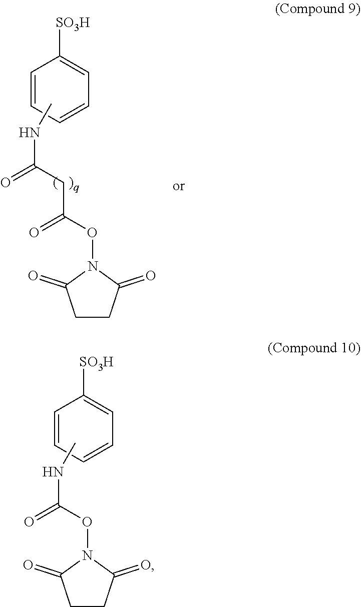

wherein, the benzenesulfonic acid is ortho-, meta- or para-substituted; and,
q is 0 to 4.

In certain embodiments, the fluorescent reagent is Compound 11, 12 or 13, their derivatives or salts thereof:

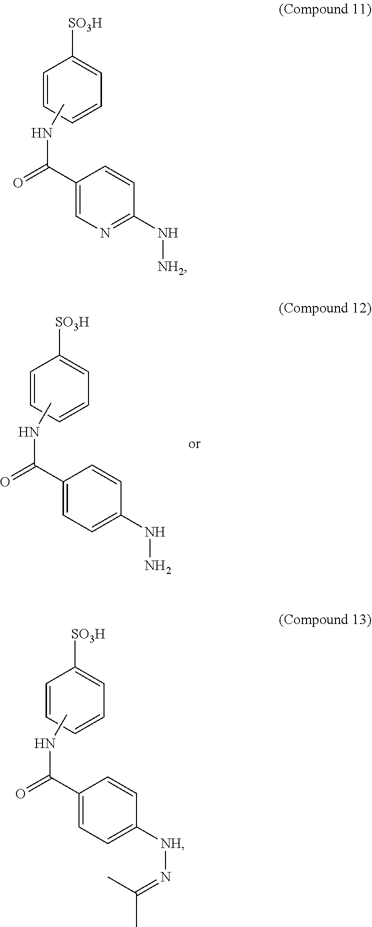

where the benzenesulfonic acid is ortho-, meta- or para-substituted.

In certain embodiments, the fluorescent reagent is Compound 6, 7 or 8, their derivatives or salts thereof:

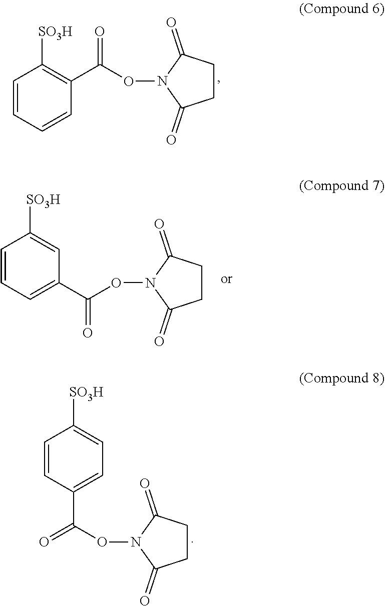 [SMILES: O=C(ON1C(=O)CCC1=O)c1ccccc1S(=O)(=O)O ; O=C(ON1C(=O)CCC1=O)c1cccc(S(=O)(=O)O)c1 ; O=C(ON1C(=O)CCC1=O)c1ccc(S(=O)(=O)O)cc1]

In certain embodiments, the aromatic ring carbocycle is labeled with ¹³C, and wherein the carbocyle contains between one and seven ¹³C atoms.

In certain embodiments, the glycoprotein is either in a solution or is immobilized on a support selected from the group consisting of a resin, a bead, a membrane, an array, a surface, a cartridge, a solid, a plate and a well.

In certain embodiments, the deglycosylation is done either by a physical method, a chemical method or by an enzyme.

In certain embodiments, the enzyme is a glycosidase.

In certain embodiments, the glycosidase is PNGase F.

Certain embodiments provide a method of detecting glycans in a sample, comprising:
(a) deglycosylating any bound glycans in the sample;
(b) contacting one or more glycans from the sample with a fluorescent reagent of Formula I:

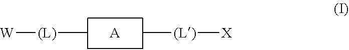

wherein,
A is an aromatic component;
X is a molecular scaffold substituent designed to react with a glycan aldehyde, ketone or an aminoglycan;
optionally attached L and L′ are linkers, that are independently (CH₂)ₙ, (OCH₂CH₂)ₘ, (CH₂)ₙ(OCH₂CH₂)ₚ or (OCH₂CH₂)ₚ(CH₂)ₙ; where n is 0 to 8, and m and p are independently 0 to 4; and,
W is a negatively charged substituent for detection;
(c) allowing the sample and the fluorescent reagent to react together to form a fluorescent target product;
(d) exciting the fluorescent target product with UV/visible light of an appropriate excitation wavelength; and,
(e) quantifying or determining the presence of the glycan in the fluorescent target product by detecting appropriate UV/visible emissions from the fluorescent target product and/or detecting the presence of the glycan in the fluorescent target product.

In certain embodiments for detecting glycans,

A is benzene, (N- or S-) substituted benzene, naphthalene, (N- or S-) substituted naphthalene, anthracene, (N- or S-) substituted anthracene, pyrene, (N- or S-) substituted pyrene, or derivatives or salts thereof;

X is an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives or salts thereof; and,

W is sulfonic acid and their derivatives or salts thereof, phosphoric acid and their derivatives or salts thereof, carboxylic acid and their derivatives or salts thereof.

In certain embodiments for detecting glycans, the activated carboxylic group is selected from: a succinimidyl, N-hydroxysuccinimidyl (NHS) ester, N-hydroxysuccinimidyl (NHS) cyanate, N-hydroxysuccinimidyl (NHS) thiocyanate, N-hydroxysuccinimidyl (NHS) isothiocyanate, and N-hydroxysuccinimidyl (NHS) isocyanate.

In certain embodiments for detecting glycans, the fluorescent reagent of Formula I is a benzenesulfonic acid derivative, or salts thereof.

In certain embodiments for detecting glycans, the fluorescent reagent is a compound of Formula II, III or IV, their derivatives or salts thereof:

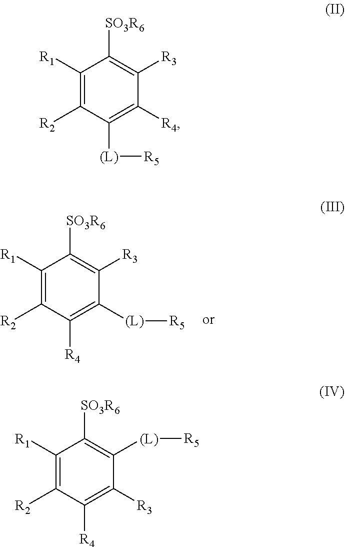

wherein,
R₁, R₂, R₃ and R₄ are independently, H, C₁-C₆-alkyl, C₁-C₆ alkoxy or halogen;
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ; where n is 0 to 8, and m and p are independently 0 to 4;
R₅ is a primary amino group, hydrazine or substituted hydrazine, hydrazide or substituted hydrazide, hydrazone or substituted hydrazone, oxime or substituted oxime, sulfonyl chloride, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds;
R₆ is H or O⁻ or OM where M is a metal ion or an ammonium ion.

In certain embodiments for detecting glycans, R₁, R₂, R₃ and R₄ are independently H, methyl, ethyl, C₃-C₆ alkyl, C₁-C₆ alkoxy or halogen.

In certain embodiments for detecting glycans, the fluorescent reagent is a compound of Formula V, VI or VII, their derivatives or salts thereof:

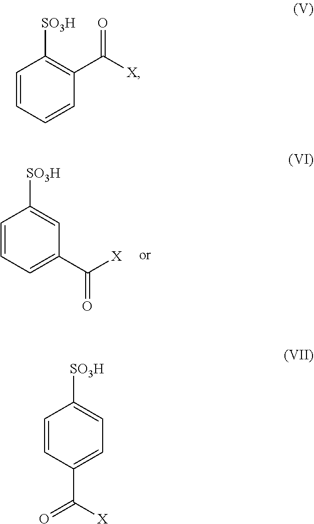

wherein, X is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof.

In certain embodiments for detecting glycans, the fluorescent reagent is a compound of Formula IX, X or XI, their derivatives or salts thereof:

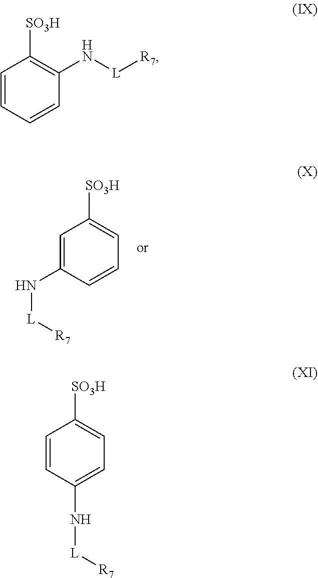

wherein,
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ where n is 0 to 8 and m and p are independently 0 to 4;
if n, m or p is 0, R₇ is H; and,
if n is 1 to 8, or if m or p are independently 1 to 4, then,
R₇ is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds.

In certain embodiments for detecting glycans, the fluorescent reagent is Compound 1, 2 or 3, its derivatives or salt thereof:

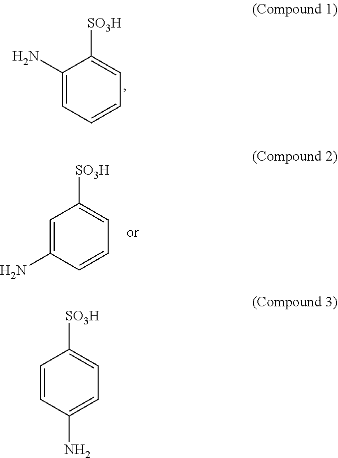 [SMILES: Nc1ccccc1S(=O)(=O)O ; Nc1cccc(S(=O)(=O)O)c1 ; Nc1ccc(S(=O)(=O)O)cc1]

In certain embodiments for detecting glycans, the fluorescent reagent is Compound 9 or Compound 10, its derivatives or salt thereof:

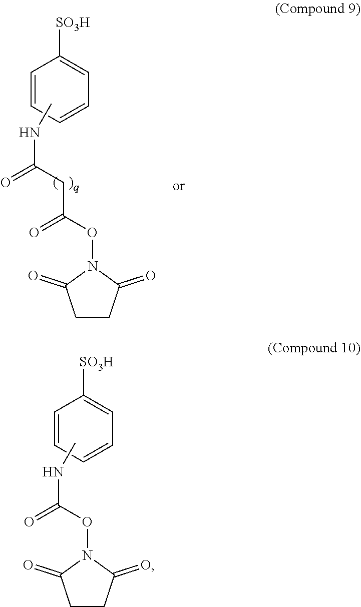

wherein, the benzenesulfonic acid is ortho-, meta- or para-substituted; and,
q is 0 to 4.

In certain embodiments for detecting glycans, the fluorescent reagent is Compound 11, 12 or 13, their derivatives or salts thereof:

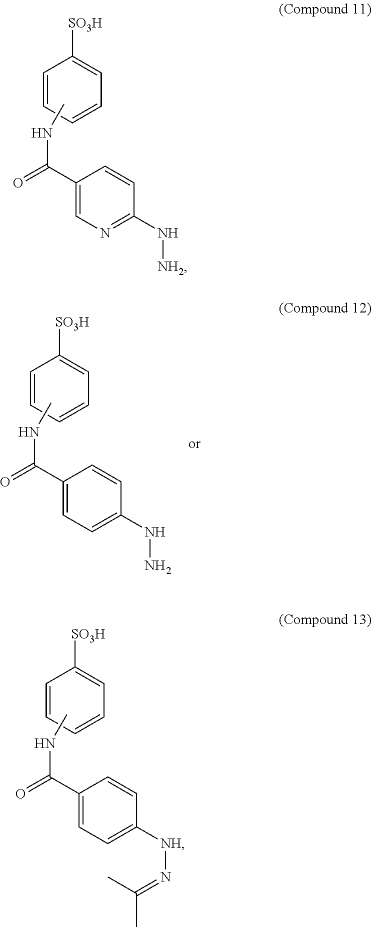

where the benzenesulfonic acid is ortho-, meta- or para-substituted.

In certain embodiments for detecting glycans, the fluorescent reagent is Compound 6, 7 or 8, their derivatives or salts thereof:

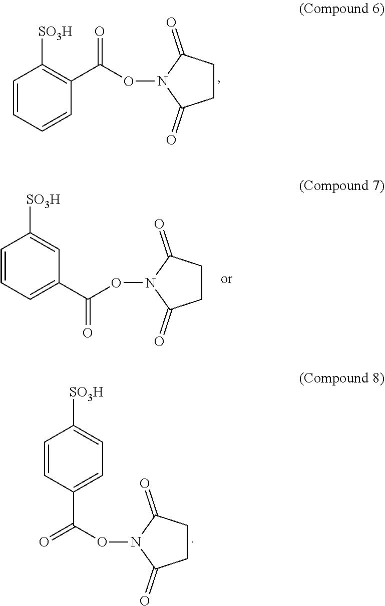 [SMILES: O=C(ON1C(=O)CCC1=O)c1ccccc1S(=O)(=O)O ; O=C(ON1C(=O)CCC1=O)c1cccc(S(=O)(=O)O)c1 ; O=C(ON1C(=O)CCC1=O)c1ccc(S(=O)(=O)O)cc1]

In certain embodiments for detecting glycans, the aromatic ring carbocycle of any one of the compounds described above is labeled with either ¹²C or ¹³C, and contains between one and seven ¹²C/¹³C atoms respectively, thereby generating ¹²C/¹³C labeled isotopalogues.

In certain embodiments for detecting glycans, the ¹²C/¹³C labeled isotopalogues are used to label glycans, and resultant ¹²C/¹³C labeled glycans are used for detection.

In certain embodiments for detecting glycans, the detecting step is determining the mass-based differences in sialic acid linkage isomers after DMT-MM derivatization.

In certain embodiments for detecting glycans, the detection is performed by MS (mass spectrometry), LC-MS, CE-MS, MS-MS, HPLC, HILIC, UPLC/UHPLC, UPLC/UHPLC-CE.

In certain embodiments for detecting glycans, the fluorescent reagent is a compound selected from the following compounds, their derivatives or salts thereof:

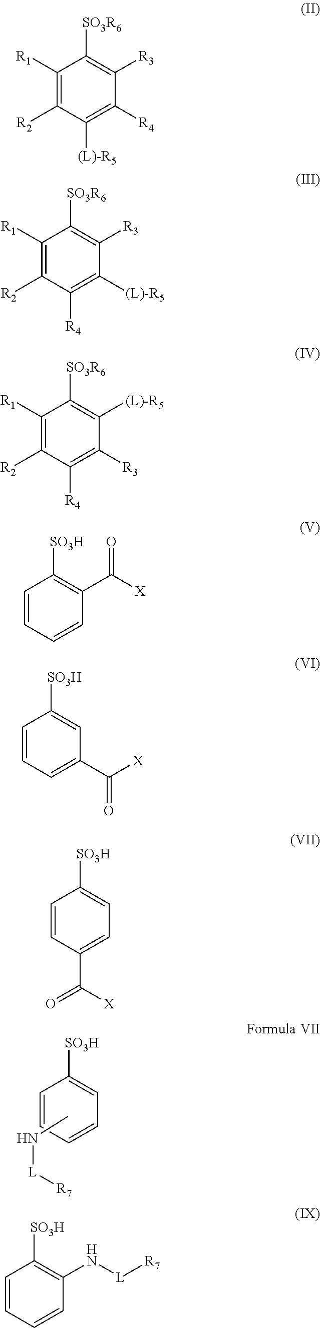

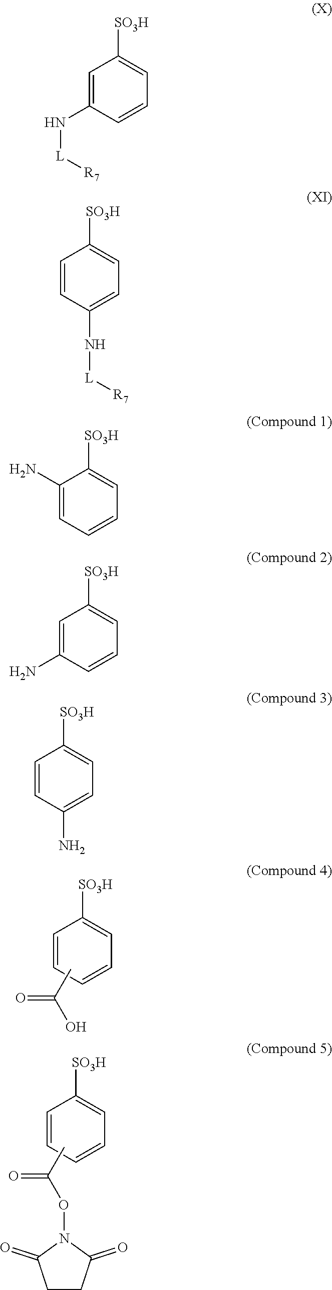

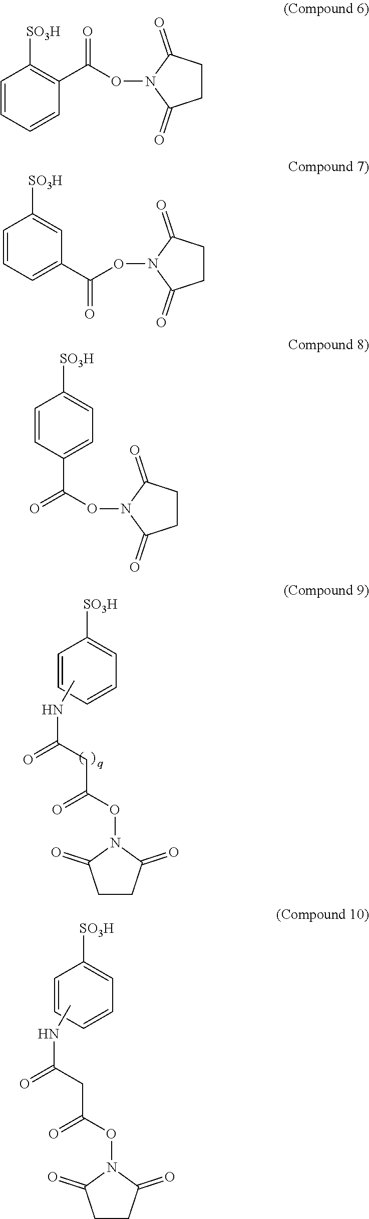

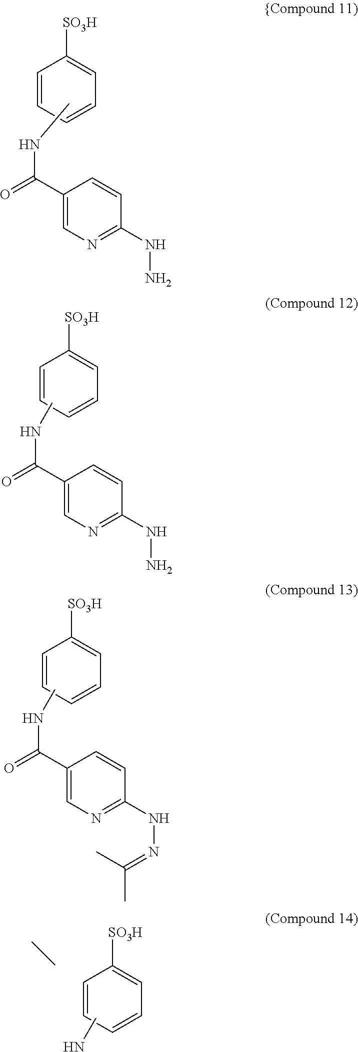

Certain embodiments for relatively quantitating glycans provide methods comprising:

a) generating ¹²C/¹³C labeled isotope analogues of any one of the fluorescent compounds of claim 35, or any one of the fluorescent compounds described in the methods claims 1-13, or the method claims 19-31 thereby resulting in a ¹²C labeled isotopologue and a ¹³C labeled isotopalogue of the selected compound;

b) generating released glycans using a glycosidase enzyme, in the first glycan sample and the second glycan sample;

c) labeling the first glycan sample with the ¹²C labeled isotopalogue of step (a) to generate ¹²C labeled glycans, and labeling the second glycan sample with the ¹³C labeled isotopalogue of step (a) to generate ¹³C labeled glycans;

d) mixing the ¹²C labeled and ¹³C labeled glycans and purifying the ¹²C/¹³C labeled glycans by removing the excess fluorescent labels;

e) coinjecting the purified ¹²C/¹³C labeled glycans into liquid chromatography or CE, and quantitating the glycans by fluorescence; and,

d) performing tandem mass spectrometry to quantitate the ¹²C/¹³C labeled glycans by mass-based differences.

In certain embodiments for relative quantitation of glycans, the method further provides

e) determining the mass-based differences in sialic acid linkage isomers after DMT-MM derivatization.

In certain embodiments for relative quantitation of glycans, the glycans are N-glycans or O-glycans.

In certain embodiments for relative quantitation of glycans the liquid chromatography is done by HILIC, UPLC/UHPLC or HPLC.

In certain embodiments for relative quantitation of glycans the mass spectrometry detection is done on a quadrupole time of flight instrument (UPLC-FLr-QToF-MS/MS).

In certain embodiments for relative quantitation of glycans the first glycan sample is an innovator biomolecule and the second glycan sample is a biosimilar biomolecule.

In certain embodiments for relative quantitation of glycans the first glycan sample is a F_{c} portion of an antibody and the second glycan sample is a F_{ab} portion of the same antibody.

In certain embodiments for relative quantitation of glycans the first glycan sample is obtained after a first treatment, and the second glycan sample is obtained after a second treatment.

In certain embodiments for relative quantitation of glycans the first glycan sample is obtained before a treatment, and the second glycan sample is obtained after the treatment.

In certain embodiments for relative quantitation of glycans the first and second glycans are samples from bioreactor runs.

In certain embodiments for relative quantitation of glycans the first and second glycans are disease samples.

In certain embodiments for relative quantitation of glycans, the samples are antibodies.

Certain embodiments provide a kit for detecting an analyte in a sample, wherein the kit comprises:

a) a compound selected from any one of the fluorescent compounds of claim 35, or any one of the fluorescent compounds described in the methods claims 1-13, or the method claims 19-31;

(b) instructions for covalently labeling the selected compound of a) to the analyte.

In certain embodiments of the kit, the analyte is a glycoprotein, a biological sample comprising a glycoprotein or part of a glycoprotein, a fusion protein or a fragment thereof, a biosimilar or a fragment thereof, an antibody or a fragment thereof, a cell or part of a cell, a virus or part of a viral protein, protein therapeutic or a fragment thereof.

In certain embodiments the kit further comprises one or more of the following: a buffering agent, a purification medium, a vial comprising the analyte, an organic solvent, one or more reagents for releasing the glycan from a biomolecule, or optionally, one or more reagents to purify the released glycan from the reaction mixture.

In certain embodiments of the kit, the reagent for releasing the glycan from a biomolecule is selected from a physical method, a chemical or an enzyme.

In certain embodiments of the kit, the enzyme is a glycosidase.

In certain embodiments of the kit, the glycosidase is PNGase F.

In certain embodiments of the kit, the instructions for detecting the analyte may also be provided.

In certain embodiments of the kit, the purification medium is selected from the group consisting of a resin, a bead, a membrane, an array, a surface, a cartridge, a solid support, a plate and a well.

In certain embodiments of the kit, the bead is a magnetic bead.

In certain embodiments the kit may further comprise instructions for labeling glycans in a sample for preparation of glycan analysis, the method comprising:

(i) treating the sample with a release reagent, such as PNGase F enzyme, with an appropriate buffer under conditions suitable for the release of the glycan from the biomolecule, thereby forming a reaction mixture;

(ii) adding beads and buffer to the reaction mixture;

(iii) separating the supernatant from the beads;

(iv) washing the beads with wash buffer;

(v) eluting the glycans from the beads with elution buffer;

(vi) performing labeling of the glycans using one or more compounds according to claim 35, thereby forming a glycan-containing solution;

(vii) optionally, removing excess compound using fresh beads; washing beads, separating the beads from excess compound/label/wash solution; and eluting glycans from the beads; and

(viii) collecting the glycan-containing solution.

### BRIEF DESCRIPTION OF THE FIGURES

A better understanding of the features and advantages of the present invention will be obtained by reference to the following figures that sets forth illustrative embodiments, in which the principles of the invention are utilized.

FIG. 1 shows a general approach to labeling glycans. Compound 15 is heated with 3-aminobenzene sulfonic acid or Compound 2, which gives Compound 17 by dehydration of intermediate Compound 16. Reduction of Compound 17 with for example, sodium cyanoborohydride or an equivalent reducing agent gives the labeled acyclic glycan Compound 18.

FIG. 2 shows more general approaches to labeling glycans with fluorescent reagents or dyes. The free amino group in Compound 19 may react with a dye-NHS carbamate to give Compound 27 or it may react with an NHS ester to give Compound 26. Its free amino group may be hydrolyzed to give Compound 20 then aldehyde Compound 21 which may react with ammonium carbonate followed by a dye NHS ester to give Compound 26 or Compound 21 may react with a hydrazide to give Compound 25. Compound 21 may undergo a reductive amination with a dye containing a free primary amino group to give Compound 22 or it may react with a hydroxylamine or a substituted hydroxylamine to afford Compound 23 or Compound 24.

FIG. 3 shows a generic carboxybenzenesulfonic acid Compound 4 undergoing carboxyl activation to the NHS esters Compound 5 (generic). The specific ortho-, meta-, and para-compounds related to Compound 5 are shown as Compounds 6, 7 and 8 respectively; any of these compounds may be used as glycan labels in any of the methods exemplified in the Examples.

FIG. 4 shows (i) the generic Formula I that may be used as a glycan label in any of the methods of the Examples; (ii) the fluorescent reagent of compounds of Formula II, III or IV that may be used as glycan labels; (iii) the fluorescent reagent of compounds of Formula V, VI or VII that may be used as glycan labels; (iv) the generic carboxy-reactive substituted aminobenzoic acid derivatives of Formula VIII and the specific ortho-, meta-, and para-compounds of Formulas IX, X and XI respectively, that may be used as glycan labels in any of the methods exemplified in the Examples.

FIG. 5 shows some generic benzenesulfonic fluorescent reagents, Compounds 9, 10, 11, 12 and 13 that may be used as glycan labels and are derived from generic aminobenzene-sulfonic acid Compound 14.

FIG. 6 shows some fluorescent reagents, namely compounds 1, 2, 3, 9, 10, 11, 12, 13 that may be used as glycan labels in any of the methods described in this disclosure.

FIG. 7A-7C shows comparison of the relative abundance of N-glycans released from human IgG (hIgG) using mass spectrometric analysis with three different dyes: A. Sulfanilic acid (3ASA); B. 2-amino benzoic acid (2AA); and C. 2-amino benzamide (2AB). Sulfanilic acid generated a smoother MS baseline curve compared to those of 2AA and 2AB.

FIG. 8A-8C shows comparison of the relative abundance of N-glycans released from human IgG (hIgG) using fluorescence detection with three different dyes: A. Sulfanilic acid (3ASA); B. 2-amino benzoic acid (2AA); and C. 2-amino benzamide (2AB). Sulfanilic acid generated fluorescence peaks with higher intensity compared to those of 2AA and 2AB.

FIG. 9 shows the relative abundance of N-glycans released from human IgG using mass spectrometric analysis with sulfanilic acid (3ASA).

FIG. 10 shows the relative abundance of N-glycans released from RNAse B using mass spectrometric analysis with sulfanilic acid (3ASA).

FIG. 11 shows the relative abundance MS profiles of N-glycans released from highly sialylated protein, AGP (human alpha 1-acid glycoprotein) using sulfanilic acid (3ASA).

FIG. 12A-12B shows the comparative analysis of the relative abundance of N-glycans released from: A. AGP (human alpha 1-acid glycoprotein)-sulfanilic acid versus B. AGP (human alpha 1-acid glycoprotein)—sulfanilic acid-DMT-MM. This technique allows for mass based differentiation of sialic acid linkage isomers by changes in structural polarity.

FIG. 13 shows sulfanilic acid labeled fragmentation of A2 glycan from AGP (highly sialylated protein).

FIG. 14 shows an overlay of three base peak intensities obtained from three separate preparations of a monoclonal antibody labeled with 3ASA.

FIG. 15 shows LC-MS method suitability for ^{12/13}C₆ 3ASA isotopic labeling based quantitation of two monoclonal antibody preparations.

FIG. 16 shows a CE electropherogram of sulfanilic acid labeled maltodextrin.

FIG. 17A-17C shows the CE-MS spectra of sulfanilic acid labeled-: A. maltotriose, B. maltotetrose, and C. maltopentose.

### DETAILED DESCRIPTION OF THE INVENTION

Glycans conjugated to proteins can affect characteristics critical to their function, including pharmacokinetics, stability, bioactivity, or immunogenicity. Several proteins, including biotherapeutics, are glycoproteins. Glycosylation can also impact drug stability, half-life, clearance in the body, immunogenicity, and other physiochemical properties. Various glycoform modifications can occur during drug design, development and optimization during bioproduction. Therefore it is important to determine the nature and types of glycans present, and analysis especially of N-linked and O-linked glycans are of particularly of interest in biological research, clinical analysis, and pharmaceutical production.

The present disclosure provides compounds that are both fluorescent and MS active for tagging glycans, such as N-linked or O-linked glycans which are useful to label and detect the glycans. Therefore, the present disclosure provides methods for labeling or binding aldehyde-, ketone-, glycosylamine-containing molecules or analytes, for e.g., glycans. The present disclosure also provides methods for comparative quantitation of glycans using the ¹²C/¹³C labeled isotopologues of the compounds described herein (including for e.g., sulfanilic acid, its derivatives or salts thereof), thereby providing indicating structural characterization and quantitation of glycan alterations. Such comparative glycomic quantitation has numerous potential applications well documented in literature; the ¹²C/¹³C labeled isotopologues of any of the compounds described herein (including for e.g., sulfanilic acid, its derivatives or salts thereof) may be applied in any such glycomic quantitation requiring application, including pharmaceutical, medical and analytical applications.

The terms glycoproteins or glycoconjugates may be used interchangeably, and comprises one or more glycans (the carbohydrate, or the saccharide, or the oligosaccharide portion, or the sugar in the molecule, which terms are meant equivalently through this disclosure, and is so understood and referenced in the art). The glycans can be either N-glycans (linked via N-linkage to the protein) or O-glycans (linked via O-linkage to the protein).

A general method for the analysis of glycans on a glycoprotein is described herein, and generally consists of one or more steps that may include:

(a) deglycosylating any bound glycans from a glycoprotein in a sample so that free glycans are obtained. The glycoprotein may be subjected to deglycosylation by, for example, in the case of N-glycans, enzymatic cleavage of the N-glycans using a glycosidase enzyme such as PNGase-F to result in free N-glycan which can be labeled. The term “bound glycan” as described herein means any glycan that is covalently attached to another molecule, for e.g., when a glycan in a glycoprotein is attached to a protein. Or, in some embodiments, a glycoprotein may be immobilized on a solid support prior to deglycosylation, facilitating separation of released glycans from the protein portion still attached to the solid immobilized support. By solid support is meant anything including a plate, resin, surface, bead, membrane, wall, layer, microarray, or any solid support that is well understood to be useful in the art. Glycans can be also be released from a glycoprotein of interest by chemical means. In general, protocols and workflows for releasing glycans from glycoproteins using deglycosylation enzymes are known in the art and one skilled in the art would be familiar with the ranges of times, temperatures and pHs used in these methods. Exemplary endoglycosidases include endo-alpha-N-acetyl-galactosaminidase, Endoglycosidase F1, Endoglycosidase F2, Endoglycosidase F3, and Endoglycosidase H. In some embodiments of the present invention, the enzyme is the well-known deglycosylation enzyme PNGase F (Peptide-N4-(acetyl-B-glucosaminyl)-asparagine amidase, EC 3.5.1.52), which releases N-glycans from the glycoprotein in the form of glycosylamines, which can then be labeled using the methods described herein. Glycosylamines are known to hydrolyze over time therefore the time between the deglycosylation step and the labeling step should be minimized. For example, N-glycans released from a glycoprotein as glycosylamines preferably may be labeled within 30 min of being released from the glycoprotein, or with shorter periods, such as about 25 min, about 20 min, about 15 min, or about 10 min, being more preferred, in that order, with “about” meaning±3 min. In preferred embodiments, the N-glycans are labeled within about 9, 8, 7, 6, 5, 4, 3, 2 or 1 min after being released from the glycoprotein, with “about” meaning±30 seconds. In some preferred embodiments, the N-glycans are labeled within 30 seconds after release from the glycoprotein.

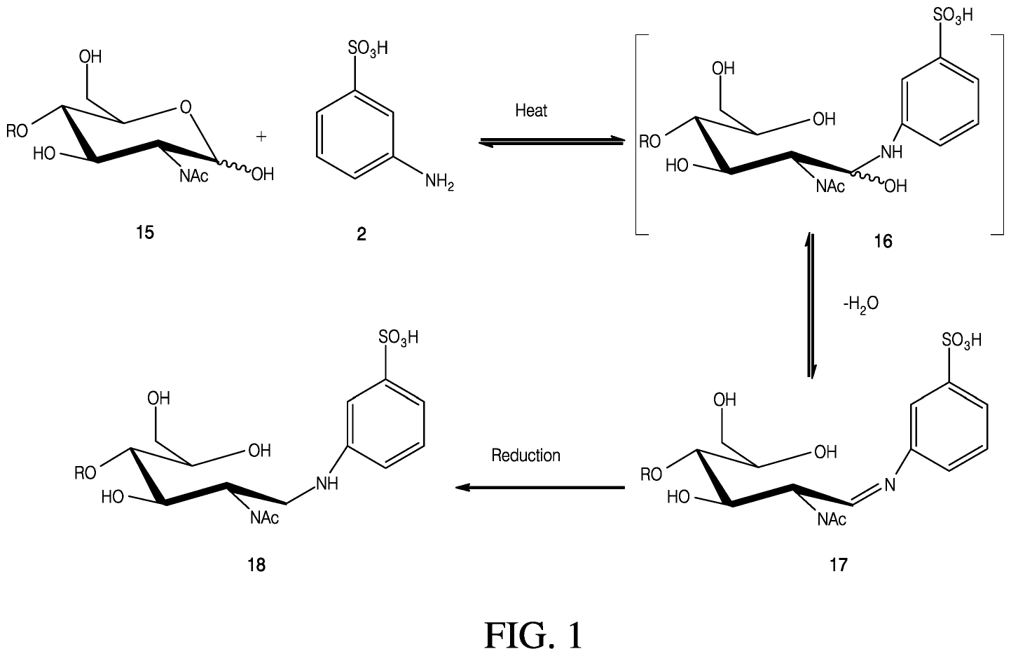

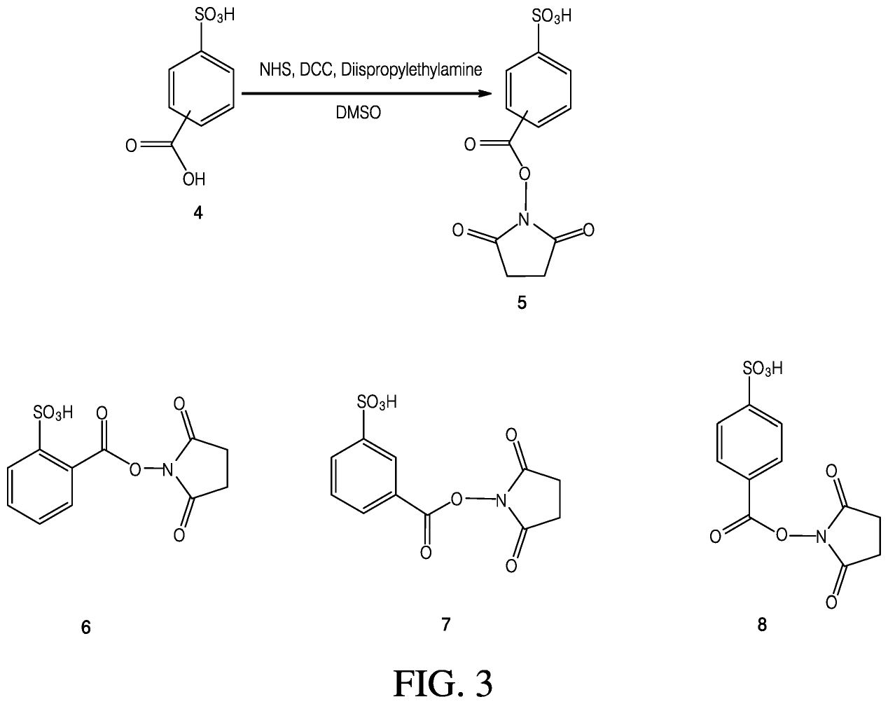

(b) The released glycans may then be labeled with a fluorescent reagent. In some embodiments, glycoproteins may be labeled, either after being released from the solid support, or while they are still immobilized. These initial sample prep steps of deglycosylation and labeling may be performed collectively either manually or via automation or in high throughput mode. Allowing enough time for labeling under appropriate conditions to permit a significant reaction to occur between a glycan sample and the fluorescent probe reagent, such as Formula (I) or sulfanilic acids or their derivatives as described in the formulae and compounds shown in FIGS. 3, 4, 5 and 6. The synthetic steps for preparing Formula (I) or sulfanilic acids or their derivatives are shown in FIGS. 1, 2 and 3. A significant labeling reaction with any of these fluorescent compounds may occur anywhere between 0.1 to 100 min, preferably at around room temperature. Reductive amination with primary or secondary amine-containing dyes, or acyl hydrazide formation with hydrazide containing dyes may require higher temperatures (for example, at around 30 to 70° C., optimally at around 50° C. for a longer time, for example, 15 to 90 min, optimally at around 30 to 60 min and with the optional use of an appropriate acid catalyst. In certain embodiments, labeling protocols may be performed with milder reagents avoiding for instance the use of sodium cyanoborohydride. Most common sample prep methods cause de-sialylation of the glycans. Preferably, the dye labeling process is optimized for minimum sialylation loss—for e.g., temperatures of 45-52° C. at 60 min gives minimum loss of sialylation. Thereafter, separation of the labeled O- or N-glycans from the excess dye (for e.g., magnetic beads may be used in this process) may be performed. Methods described herein allow rapid labeling of O- and N-glycans and more generally, primary and secondary amine-containing compounds and aldehyde/ketone/glycosylamine containing compounds under relatively mild conditions. One skilled in the art would be familiar with methods for labeling N-glycans under mild conditions using other compounds. U.S. Pat. Nos. 8,124,792 and 8,445,292, for example, discuss labeling of glycans under mild conditions. Labeling between free glycans and the compounds described herein via reductive amination may be conducted at about 10 to 60° C., preferably at 45 to 55° C., and more preferably at about 50° C., with “about” meaning±3° C. and most preferably at 50° C. Labeling aminoglycans with NHS ester probes is preferably conducted at room temperature at around about pH 7 to about pH 9, more preferably pH 7.5 to pH 8.5, and still more preferably, at about pH 8, with about meaning±0.25 pH units. Most preferably, the pH is about 8.0. For e.g., reductive amination and hydrazine/hydrazide reactions may be done in more acidic media (around pH 3 to 5) conditions at higher temperatures (around 50° C.).

(c) The resultant labeled glycan (labeled for e.g., using the formulae or compounds described in FIGS. 3, 4, 5 and 6 herein) may be highly fluorescent, thereby providing excellent detection of the labeled glycan in solution. The resultant mixture of N-labeled glycans from a given sample may be separated from each other by liquid chromatography, such as high performance liquid chromatography (HPLC), HILIC (hydrophilic interaction liquid chromatography), or ultra-high performance liquid chromatography (UPLC), or they may be separated by using capillary electrophoresis (CE), or a combination of CE-LC. Quantitative or qualitative detection of the amount of the separated, labeled glycan may be done a fluorescent reporting signal from the labeled glycans. This process may use UV/visible, or fluorescence detection using appropriate excitation and emission wavelengths. The resultant labeled glycan (labeled for e.g., using the formulae or compounds described herein in FIGS. 3, 4, 5 and 6), may also be highly charged thereby enhancing its detection in negative ion mass spectrometry (MS). Following LC or CE, mass spectrometry detection may be used for structural elucidation of the types of glycans present in a sample. In one exemplary embodiment for instance, the N- labeled glycans may be separated from each other by chromatography, such as high performance liquid chromatography (LC, HPLC), or ultra-high performance liquid chromatography (UPLC, UHPLC, HILIC, etc.), or by using capillary electrophoresis (CE). Generally, for LC based separation techniques, sialylated glycans elute at the end of the LC profile resulting in poor resolution; whearas in CE based separation techniques, highly sialylated glycans migrate at the beginning of the electropherogram, enabling accurate quantitation.

In an embodiment exemplified in the examples and in the Figures herein, one or more of techniques may be combined for e.g., multiplexing may be performed with UPLC-fluorescence—MS analysis, CE-LC, LC-MS, CE-MS, MS-MS, etc. This is sometimes referred to as twoplexed/duplexed/multiplexed analysis, and is useful for detailed structural elucidation of glycoforms present in a sample. These combinatorial techniques may be set up for automation. In certain embodiments for duplex/multiplex analysis, the formulae or compounds described in FIGS. 3, 4, 5 and 6, or commonly referred to as sulfanilic acids (SA) can be synthesized as ¹³C/¹²C isotopologues to generate relative MS data that helps in quantitation and detailed glycan structural analysis. In certain embodiments, the formulae or compounds described herein, or commonly referred to as sulfanilic acids (SA), are used for determining specific sialic acid linkages after treatment with the condensation reagent DMT-MM. DMT-MM is 4-(4,6-Dimethoxy-1,3,5-triazin yl)-4-methylmorpholinium chloride. DMT-MM derivatization allows for mass-based differentiation of sialic acid linkage isomers and their simultaneous and differential glycomic analysis because of improved HILIC (Hydrophilic Interaction Liquid Chromatography) separation mediated by changes in their structural polarity. Such methods, when combined with the use of ¹³C isotopologue-labeling on the reducing terminus of glycans/saccharides, may enable the differential analysis and/or the quantitation of (¹²C/¹³C) individual glycan/saccharide isomers within complex mixtures of glycans/saccharides, such as in biological samples and/or medical samples, etc. The methods described herein are well understood and described in the art; for e.g., in Ruhaak et al., Anal Bioanal Chem. (2010); August; 397(8): 3457-3481; Albrecht et al., Proteomics (2017); 17, 1-2; Varadi et al., Anal. Bioanal. Chem., 2016 December; 408(30):8691-8700: all of which are incorporated by reference in entirety.

This type of analysis may be useful in studying glycosylation patterns of any biological material, or any medical sample, including but not limited to cancer, metabolic disease samples; for assessing the critical quality attribute (CQA) of a glycosylated protein therapeutics; for quantitative glycomics; for evaluating biological activity, quality/purity and efficacy during bioproduction, including antibodies, protein therapeutics; for comparing innovator and biosimilar glycosylated therapeutic molecules; for selecting therapeutic molecules with a desired glycosylation; to distinguish between Fc and Fab fragments, etc. Nearly 40% of all approved therapeutic proteins are glycosylated, and 8 out of the 10 top selling protein therapeutics contain N-linked oligosaccharides. Glycosylation has an impact on drug stability, half-life, clearance (dose repeats), immunogenicity, and other physiochemical properties. Glycoform modifications can occur during drug design, development and optimization during bioproduction. Critical quality attributes (CQA) may be strictly monitored during bioproduction of biotherapeutics. The consistency of the production process, and changes in glycosylation patterns and levels can be monitored by the glycan profiles.

Following labeling, aqueous media, for example, water, may be added to dilute the reactants to an appropriate range before taking sample analytes to be introduced into an analytical instrument or instruments by which the practitioner intends to analyze the labeled compound or compounds. Analyses, such as measuring fluorescent intensity and performing mass spectrometry are commonly performed in the art and one skilled in the art would be familiar with preparing samples of N-glycans and other compounds for such analysis, including bringing the samples up to pre-determined equal volumes before introducing an aliquot into the instrument. For example, after labeling, volumes of each replicate were brought up to 100 μL before an aliquot was taken and introduced into the analytical instrumentation, but the volume may depend on the quantity of initial reactants and on the particular system on which the labeled compounds are to be analyzed. A dilute acid, such as dilute acetic acid or formic acid, may be used in place of water to bring the volume up to the pre-determined volume.

Compounds

The present disclosure provides compounds that are both fluorescent and MS active. The compounds may be used in labeling or tagging a target analyte, for e.g. a glycan of a glycoprotein and subsequently, in the separation of the labeled glycans and/or detection by a variety of methods. In certain variations, the compounds described herein can be further modified, and the modifications may facilitate rapid labeling, better fluorescence detection, better mass spectrometry detection, and/or enable the translation of compounds into kits described below. In general, the compounds comprise (a) at least one fluorescent group such as an aromatic, substituted aromatic ring, aromatic, a phenyl, a polycyclic aromatic, a heterocycle, (b) at least one group capable of binding to an aldehyde or ketone present on an analyte of interest, for e.g.; a glycoprotein; and (c) at least one negatively charged group that can be detected by negative ion mass spectrometry.

Therefore, the present disclosure provides compounds that are both fluorescent and MS active, having a general formula (I):

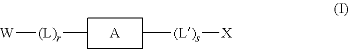

wherein,
A is a molecular scaffold containing an aromatic component which is fluorescent;
X is a substituent designed to be able to react with glycan aldehydes, or glycan ketones, or glycosylamines;
W is a negatively charged substituent for detection; and
independent linkers L and L′ that can be attached optionally.

Accordingly, A, the molecular scaffold can comprise an aromatic group including but not limited to benzene, (N- or S-) substituted benzene, naphthalene, (N- or S-) substituted naphthalene, anthracene, (N- or S-) substituted anthracene, pyrene, (N- or S-) substituted pyrene, derivatives or salts thereof of any of the preceding compounds.

Accordingly, X may be a group for coupling to glycan aldehydes, or glycan ketones, or a glycosylamine, for e.g., an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds.

Accordingly, W may be a negatively charged group designed to enhance detection in negative ion mass spectrometry (MS), including but not limited to, sulfonic acid and their derivatives or salts thereof, phosphoric acid and their derivatives or salts thereof, carboxylic acid and their derivatives or salts thereof.

Throughout the present disclosure, by ‘composition(s)’ or ‘compound(s)’ is meant, generally, a compound have the general structure of (I), and in a preferred embodiment, having the structure of sulfanilic acid (SA) and their derivatives or salts thereof. However, by ‘composition(s)’ or ‘compound(s)’ is also meant, any compound/formula as described in Formula I, II, II, IV, V, VI, VII, VIII, IX, X, XI, their derivatives or salts thereof, as shown in FIG. 3, 4, 5 or 6; or any compound described as Compound 1, 2, 3, 9, 10, 11, 12, 13, their derivatives or salts thereof, as shown in FIGS. 3, 4, 5 and 6. Even though the disclosure may generically refer to sulfanilic acid singularly in certain instances, or to sulfanilic acid and its derivatives, or in exemplary embodiments, this disclosure is meant to include any of the Formulae I, II, II, IV, V, VI, VII, VIII, IX, X, XI, their derivatives or salts thereof, as shown in FIG. 3, 4, 5 or 6; or to mean any of the Compounds of 1, 2, 3, 9, 10, 11, 12, 13, their derivatives or salts thereof, as shown in FIGS. 3, 4, 5 and 6, any of which would work equivalently in the embodiments. Most of these compounds would have a permanently negatively charged label and/or a negative ion polarity for example, thereby making them MS active. Most of these compounds would also be fluorescent. Also, certain terms in the claims or in the disclosure refer to the compositions or compounds as: label(s), tag(s), fluorescent compound(s), fluorescent tag(s), fluorescent label(s), MS active compound(s), MS active label(s), MS active tag(s), etc.—these too are meant to refer to sulfanilic acid and its derivatives; to any of the Formulae of I, II, II, IV, V, VI, VII, VIII, IX, X, XI, their derivatives or salts thereof, as shown in FIG. 3, 4, 5 or 6; to any of the Compounds of 1, 2, 3, 9, 10, 11, 12, 13, their derivatives or salts thereof, as shown in FIGS. 3, 4, 5 and 6, any of which would work equivalently in the embodiments. In certain exemplary embodiments, sulfanilic acid or aminobenzene sulfonic acid which includes all forms 2ASA, 3ASA and 4ASA may be used in the methods and kits described here. 3-sulfanilic acid or 3ASA, is an exemplary embodiment used for demonstration of the principles underlying this class of compounds. The skilled artisan would understand that any of the above described compositions, compounds, tags, or labels may be used instead of 3-sulfanilic acid or 3ASA or aminobenzene sulfonic acid for the application. The skilled artisan would also understand that any of the compound described here are fluorescent and/or MS active and therefore are applicable for protocols, methods, kits, uses, etc. discussed throughout the disclosure. Accordingly, the skilled artisan would understand that any of the above described compositions, compounds, tags, labels may be used in tagging/labeling a target analyte, or tagging/labeling a biological sample, or tagging/labeling a glycoprotein, or tagging/labeling a glycoconjugate, or tagging/labeling a glycan, or tagging/labeling an N-glycan, etc. The term ‘derivative’ is well understood in the art. The term ‘salts’ may include but are not limited to a potassium, sodium, ammonium, alkylammonium, tetralkylammonium, triethylammonium, tert-butylammonium, tetralkyl-phosphonium, tetraaryl-phosphonium, lithium or cesium ion.

In exemplary embodiments, stable isotope variants of sulfanilic acid—¹³C₆ isotopologue may be generated, which provide superior quantitative and comparative MS/MS based analyses. Similarly, stable ¹³C₆ isotopologues of the compositions/compounds described above (for e.g, any of Formulae I, II, II, IV, V, VI, VII, VIII, IX, X, XI, their derivatives or salts thereof, as shown in FIG. 3, 4, 5 or 6; or for e.g., for any of the compounds of 1, 2, 3, 9, 10, 11, 12, 13, their derivatives or salts thereof,) can also be generated for quantitative and comparative MS/MS based analyses.

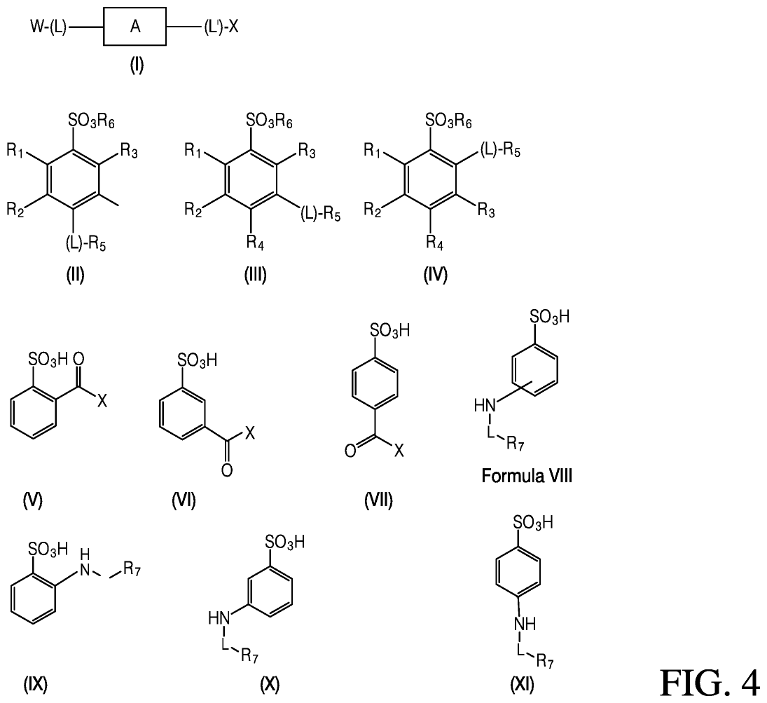

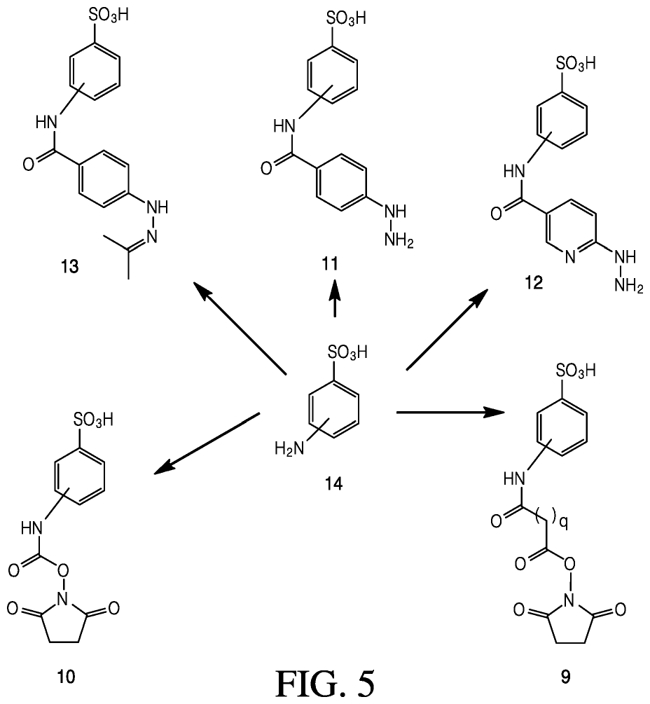

Thus in certain embodiments, described herein are compositions comprising the compounds as shown in FIGS. 4 and 5, namely, the carboxy-reactive substituted aminobenzoic acid derivatives of Formula VIII, and the specific ortho-, meta-, and para-derivative compounds of Formulas IX, X and XI, respectively; or the benzenesulfonic fluorescent reagents of Compounds 9, 10, 11, 12 and 13. In certain embodiments, the compound is a salt. In some embodiments, the salt comprises a potassium, sodium, ammonium, alkylammonium, tetralkylammonium, triethylammonium, tert-butylammonium, tetralkyl-phosphonium, tetraaryl-phosphonium, lithium or cesium ion.

Accordingly, the compounds or compositions disclosed herein are fluorescent, MS active compounds that comprise three functional components: (a) a molecular scaffold comprising an aromatic group (for fluorescence), including but not limited to benzene, (N- or S-) substituted benzene, naphthalene, (N- or S-) substituted naphthalene, anthracene, (N- or S-) substituted anthracene, pyrene, (N- or S-) substituted pyrene, derivatives or salts thereof of any of the preceding compounds; (b) a group for coupling to glycan aldehydes, or glycan ketones, or a glycosylamine, for e.g., an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds; (c) a negatively charged group designed to enhance detection in negative ion mass spectrometry (MS), including but not limited to, sulfonic acid and their derivatives or salts thereof, phosphoric acid and their derivatives or salts thereof, carboxylic acid and their derivatives or salts thereof.

In some embodiments, the compounds or compositions disclosed herein may further comprise one or more linkers which may be optionally placed between the aromatic substituent or component (A) and the negatively charged substituent or component (W); and/or, one or more linkers may be optionally placed between the aromatic substituent or component (A) and the molecular scaffold substituent or component designed to react with a glycan aldehyde or ketone (X) (see Formula (I), FIG. 4). Accordingly, the linker L′ may be (CH₂)ₙ, (OCH₂CH₂)ₘ, (CH₂CH₂O)ₘ, (CH₂)—(OCH₂CH₂)ₚ or (OCH₂CH₂)ₚ(CH₂)ₙ where n is 0 to 8 and m and p may be independently 0 to 4. In other embodiments, when more than one linker is present in the compound, the optionally attached linkers L and L′, independently, may be (CH₂)ₙ, (OCH₂CH₂)ₘ, (CH₂)—(OCH₂CH₂)ₚ or (OCH₂CH₂)ₚ(CH₂)ₙ; where n is 0 to 8, and m and p may be independently 0 to 4, (see for e.g., Formula (I), FIG. 4). In yet other embodiments, the optionally attached linker L may be (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ where n is 0 to 8, and m and p independently are 0 to 4; and wherein if n, m or p is 0, R₇ is H; and if n is 1 to 8 or if m or p are independently 1 to 4 then R₇ is selected from NH₂, a carbamate group, a hydrazine, substituted hydrazine, hydrazide, oxime or substituted oxime, aminooxime, sulfonyl chloride, a carboxylic acid or activated carboxylic group, a succinimidyl, N-hydroxysuccinimidyl (NHS) ester or equivalents including isothiocyanate, isocyanate, aminoglycans to form amides, ureas or thioureas.

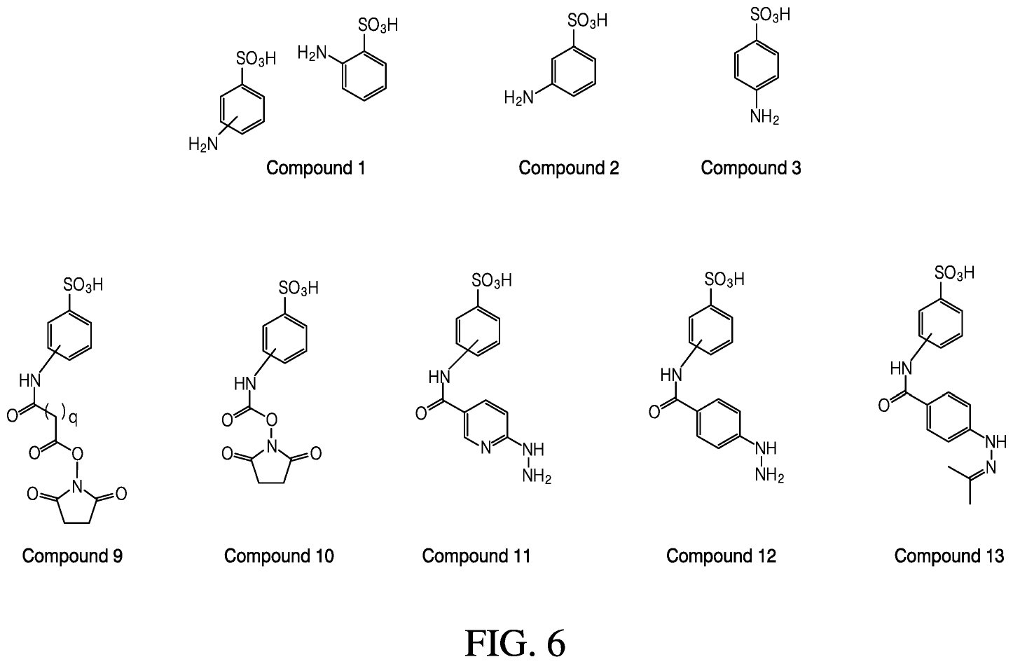

In an illustrative embodiment, sulfanilic acid (SA) or 3-aminobenzene sulfonic acid may be used to label N-glycans released from glycoproteins through standard reductive amination in the presence of reducing agents. As discussed above, similar methods may be applied to any of the compounds shown in FIG. 3 (for e.g., compounds 6, 7, or 8), FIG. 4 (for e.g., formulae I, II, III, IV, V, VI, VII, VIII, IX, X, XI), FIG. 5 (for e.g., compounds 9, 10, 11, 12 and 13), and FIG. 6 (for e.g., compounds 1, 2, 3, 9, 10, 11, 12 and 13), for N-glycan labeling.

In an illustrative embodiment, the permanent negative charge imparted by the sulfonic acid group can make sulfanilic acid and its derivatives ideal labeling reagents for negative ion mode N-glycan analysis. When compared to the current labeling solutions with permanent positive charges, the use of a permanently negatively charged label and negative ion polarity, such as seen with sulfanilic acid and its derivatives, is highly attractive for its substantially higher analytical benefits to the end user. Some of the reasons for the superior analytical benefits of sulfanilic acid, its derivatives/salts, or its equivalents (described above) are outlined here: (i) the permanently negatively charged label in the negative ion mode generates a cleaner MS and MS/MS spectra due to the lower overall ion flux into the instrument compared to positive ion mode which, although ion counts are generally one order of magnitude lower than positive ion mode, resulting in a more sensitive analyses due to the removal of background ions; (ii) the permanently negatively charged label facilitates the analysis of sialylated N-glycans, since the ionization bias and then instability of sialylated oligosaccharide ions encountered typically during analysis with positive ion polarity is removed. More importantly, increased ionization efficiency of sialylated glycans is achieved, which further increases the overall analytical sensitivity of N-glycans, for e.g, sialylated glycans and associated oligosaccharides; (iii) highly informative MS/MS analysis may be possible with N-glycans labeled with sulphanilic acids and its derivatives. The application of MS/MS with negative ion polarity may result in both glycosidic cleavages and cross ring fragmentation, which may facilitate more informed structural annotation of the N-glycans; (iv) monosaccharide rearrangements, in particular fucose migration, which is typically problematic during MS/MS glycan analysis using positively charged amine containing labels, have not been observed with sulfanilic acids and its derivatives; (v) issues with intramolecular analyte interactions and problems with selectivity on hydrophilic interaction and weak anion exchange phases, especially seen with sialylated N-glycans using the positively charged labels, for e.g., procainamide based labels like the RapiFlour MS reagent, are avoided when permanently negatively charged labels like sulfanilic acid and its derivatives are used. When using permanently negatively charged labels like sulfanilic acid and its derivatives, complications associated with the use of certain exoglycosidase enzymes for monosaccharide and linkage anomericity assessment have also been avoided; (vi) sulfanilic acid and its derivatives can be synthesized as ¹³C₆ isotopologues based on the sulphonation of ¹³C₆ aniline by fuming sulphuric acid to generate a solution for relative MS based quantitation and comparability analysis. Such ¹³C₆ isotopologues generation is not possible using for e.g., RapiFluor MS; (vii) Sulfanilic acid and its derivatives may be compatible with DMT-MM based sialic acid linkage specific derivatization chemistry which may be suitable for both LC-MS and CE-MS based glycan analysis. This method, when combined with the use of ¹³C₆ isotopologue-labeling on the reducing terminus of glycans/saccharides, may enable the differential analysis and/or quantitation of (¹²C₆/¹³C₆) individual glycan/saccharide isomers within a complex mixture of glycans/saccharides; (viii) the additional negative charge imparted on the labeled glycans due to labeling with sulfanilic acid and its derivatives may be beneficial in chromatographic purification methods, for e.g., using mixed mode, ion-exchange, anion exchange, or HIC columns.

Quantitative glycomics represents an actively expanding research field ranging from the discovery of disease-associated glycan alterations to the quantitative characterization of N-glycans on therapeutic proteins. Commonly used analytical platforms for comparative relative quantitation of complex glycan samples include MALDI-TOF-MS or chromatographic glycan profiling with subsequent data alignment and statistical evaluation.

Sulfanilic acid (or its derivative/salts, or its equivalent) labeled N-glycans provide high sensitivity for UPLC/UHPLC-fluorescence, and informative negative ion polarity with strong [M-2H]²⁻ pseudomolecular ions suited for LC-MS and CE-MS analysis of complex oligosaccharides. Additionally, a stable isotope variant of sulfanilic acid—¹³C₆ isotopologue may be generated, which provided superior quantitative and comparative MS/MS based analyses. The stable ^{12/13}C₆ sulfanilic acid isotopologues are useful for quantitative glycan profiling in a twoplex manner, which generates extracted ion electropherograms (EIE) for ¹²C₆‘light’ and ¹³C₆ ‘heavy’ sulfanilic acid labeled glycan isotope clusters. The twoplex quantitative CE-MS glycan analysis platform is ideally suited for comparability assessment of biopharmaceuticals, such as monoclonal antibodies, for differential glycomic analysis of clinical materials, for potential biomarker discovery, or for quantitative microheterogeneity analysis of different glycosylation sites within a glycoprotein. Other glycomic applications include applications such as studying glycosylation patterns of biological or medical samples, or for assessing protein production, protein quality/purity, for comparing innovator and biosimilar glycosylated proteins, or for selecting proteins with the desired glycosylation.

Labeling, Analyzing and Quantitation with Sulfanilic Acid or its Derivatives

In an exemplary embodiment, the labeling of the analyte, for e.g., N-glycan was done with sulfanilic acid or any of its derivatives (described herein in FIGS. 3, 4, 5, and 6), followed by a total analytical solution by LC-MS or CE-MS. In certain variations, the sulfanilic acid can be modified with simple NHS linker groups. The modifications may facilitate rapid labeling, may facilitate better fluorescence detection, may facilitate better mass spectrometry detection, and/or enable the translation of compounds into kits described below. For instance, in kits, magnetic beads may be employed for the removal of residual labeling reagent, or to facilitate sample clean-up prior to the analysis, further facilitating compatibility with manual or automated sample prep workstations. Additionally, the use of ^{12/13}C isotopologues of sulfanilic acids and its derivatives, can be useful in applications for the differential analysis and/or quantitation of (¹²C/¹³C) isotopologues individual glycan/saccharide isomers within a complex mixture of glycans/saccharides. Additionally, the permanent negative charge on sulfanilic acid and its derivatives makes it suitable of negative ion based labeling of glycans. The permanent negative charge on sulfanilic acid offers an improvement in electrophoretic mobility and CESI-MS analysis compared to the current offerings of APTS, 2AA and 2AB, which show lower fluorescence than the single negatively charged sulfanilic acid tag.

Sialylation of Glycoproteins

Terminal sialylation of glycosylated polypeptides has been reported to increase serum-half life of therapeutics. In the biotechnological production of therapeutic polypeptides such as immunoglobulins the assessment of oligosaccharide microheterogeniety and its batch-to-batch consistency are important tasks. Monoclonal antibodies (mAbs) are one of the fastest growing classes of protein therapeutics. Antibodies differ significantly from other recombinant polypeptides in their glycosylation pattern. Immunoglobulin G (IgG) for e.g., is a symmetrical, multifunctional glycosylated polypeptide of an approximate molecular mass of 150 kDa consisting of two identical Fab parts responsible for antigen binding and the Fc part for effector functions. Glycosylation tends to be highly conserved in IgG molecules at Asn-297, which is buried between the CH₂ domains of the Fc heavy chain, forming extensive contacts with the amino acid residues within CH₂ (Sutton and Phillips, Biochem. Soc. Trans. 11 (1983) 130-132). The oligosaccharides bound to the Fc region, do not only effect physicochemical properties (e.g. structural integrity) and abolish or minimize protease resistance but are also essential for effector functions, such as complement binding, binding to macrophage Fc receptors, rapid elimination of antigen-antibody complexes from the circulation, and induction of antibody-dependent cell-mediated cytotoxicity (ADCC) (Cox, K. M., et al., Nature Biotechnol. 24 (2006) 1591-1597; Wright and Morrison, Trends Biotechnol. 15 (1997) 26-32). Because different glycoforms can be associated with different biological properties, the ability to enrich for a specific glycoform may be useful, for example, to elucidate the relationship between a specific glycoform and a specific biological function. Thus, production of glycosylated polypeptide compositions that are enriched for particular glycoforms is highly desirable.

Kits

Conveniently, one or more of the materials required for methods described herein can be provided in kit form either in dried form or in solution. This may be added directly to a sample of the compound analyte which is to be labeled. The kits may further provide instructions for labeling an amine-containing compound or an aldehyde/ketone of interest. For example, the kits may include directions on how to label a N-glycan. The kit may also include reagents, such as PNGase F or one or more other deglycosylation enzymes, to facilitate release and labeling of glycans from a glycoprotein.

In certain embodiments, the kit further comprises instructions for covalently bonding the compound to the analyte. In certain embodiments, the kit further comprises one or more of the following: a buffering agent, a purification medium, a vial comprising the analyte, or an organic solvent, one or more reagents for releasing the glycan from a biomolecule, and optionally, one or more reagents to purify the released glycan from the reaction mixture.

The kit may further comprise instructions for labeling glycans in a sample in preparation of glycan analysis, the method comprising: treating the sample with a release reagent, such as PNGase F enzyme, with an appropriate buffer under conditions suitable for the release of the glycan from the biomolecule, thereby forming a reaction mixture; adding beads and buffer to the reaction mixture; separating the supernatant from the beads; washing the beads with wash buffer; eluting the glycans from the beads with elution buffer; performing dye labeling of the glycans using one or more dye compounds provided herein, thereby forming a glycan-containing solution; optionally, removing excess dye using fresh beads; washing beads, separating the beads from excess dye/wash solution; and eluting glycans from the beads; and collecting the glycan-containing solution. In certain embodiments, the glycan solution may be stored for future use according to instructions provided, or analyzed for its glycan profile using a CE analyzer or UPLC analyzer or a combination thereof, and/or, using UV/visible or fluorescence detection in appropriate excitation and emission wavelengths, and/or, preceding or following the previous techniques with mass spectrometry detection, which may be used for structural elucidation of at least one glycan present in a sample.

The present disclosure provides compounds comprising (a) at least one fluorescent group such as an aromatic or substituted aromatic ring; (b) at least one group capable of binding to an aldehyde or ketone present on an analyte of interest, for e.g.; to a glycoprotein; and (c) at least one negatively charged group that can be detected by negative ion mass spectrometry. The resultant compound, for e.g., the labeled glycan, may be highly fluorescent, thereby providing an excellent method for detection of aldehydes, ketones or glycosylamines in solution, and additionally, may be highly charged thereby enhancing the detection in negative ion mass spectrometry (MS) detection. The compounds can also be analyzed using capillary electrophoresis (CE), liquid chromatography (LC, UPLC, UHPLC, etc.), other similar techniques. The above techniques can also be twoplexed/duplexed/multiplexed for analysis, for e.g., UPLC-fluorescence—MS analysis, or CE-LC, or LC-MS, or CE-MS, or MS-MS, etc. These techniques or their combinations may be set up for automation.

Certain embodiments provide the use of such compounds, compositions or kits for labeling an analyte. In certain embodiments, the analyte is a biological molecule; for e.g. glycans or glycoproteins. In specific embodiments, the compounds or compositions described can label glycans.

The kits described herein for labeling glycans comprise the compounds generically described in formula (I) and specifically described in FIGS. 3, 4, 5 and 6.

### EXAMPLES

Synthesis of NHS Ester of Sulfobenzoic Acid. NHS Ester of sulfobenzoic acid can be synthesized essentially by adopting the method of Navotny et al (Journal of Proteome Research, 6 (2007) 124-132). Briefly, to a solution of sulfobenzoic acid in dry DMSO was added 1.5 molar excess of dicyclohexylcarbodiimide. This solution was stirred at room temperature for approximately 10-15 min, after which equimolar amounts of N-hydroxysuccinimide and diisopropylethylamine were added to the reaction mixture. The reaction mixture was stirred at room temperature for 3 h. The precipitated dicyclohexylurea, a side product of the reaction, was filtered and discarded. The remaining solution was then allowed to react for 2 h and was again filtered to remove the side product. After the second filtration, approximately 60 mL of cold acetone (4° C.) was slowly added to precipitate the product. The final product was then washed four times; each washing step involved the addition of approximately 15 mL of cold acetone. After the final washing, the product was dried under vacuum for approximately 2 h to give the desired product.

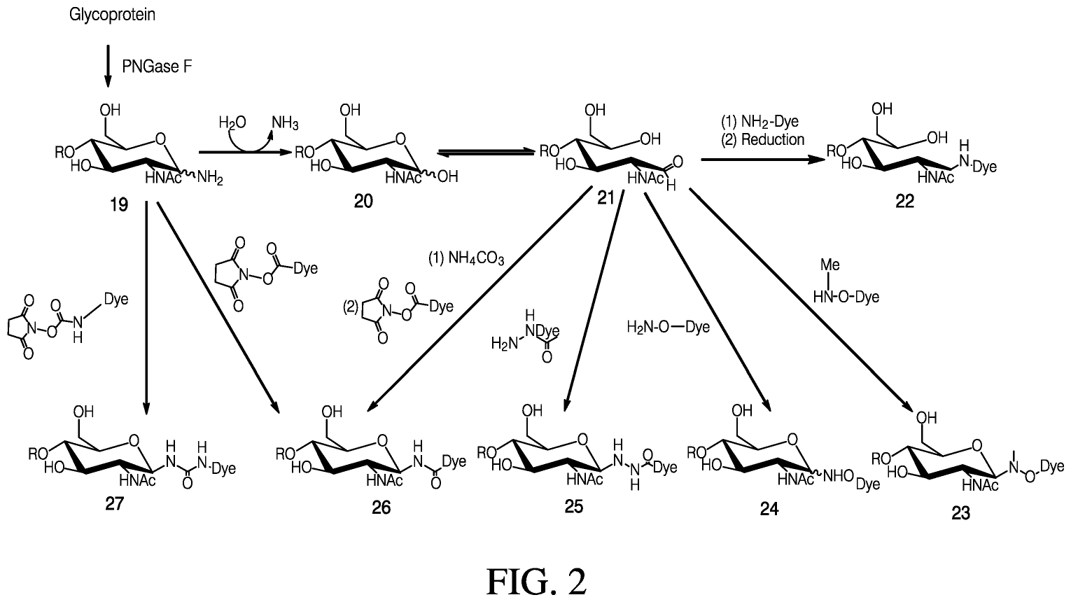

NHS Conjugation (Reaction Scheme of FIG. 2): Conjugation with NHS dyes can be achieved through the addition of 10 ml of ˜100 mM dye solution in DMSO to 20-50 ml of glycans released from glycoproteins (5-200 mg) in the presence of basic buffers (100-200 mM sodium bicarbonate buffer, ˜pH 8 or HEPES buffer 100-200 mM sodium bicarbonate buffer, ˜pH 8) and incubating at ambient temperature 10-30 minutes. Sample was diluted 5-10 fold with acetonitrile-water (75:25 v/v) prior to injection.

Reductive Amination (Reaction Scheme of FIG. 1): Aminobenzene sulphonic acid derivatization reactions were performed adding 2 μl of ˜0.3M solution of the dye in acetic acid-DMSO (3:7 v/v) to a 1-100 nmoles of pure glycans or a mixture of glycans released from glycoproteins (5-200 μg) in water followed by 2 μl of 1M reductant in DMSO. After mixing the contents the tube was incubated for 1-2 hrs at 50° C. Sample was diluted 5-10 fold with acetonitrile-water (75:25 v/v) prior to injection.

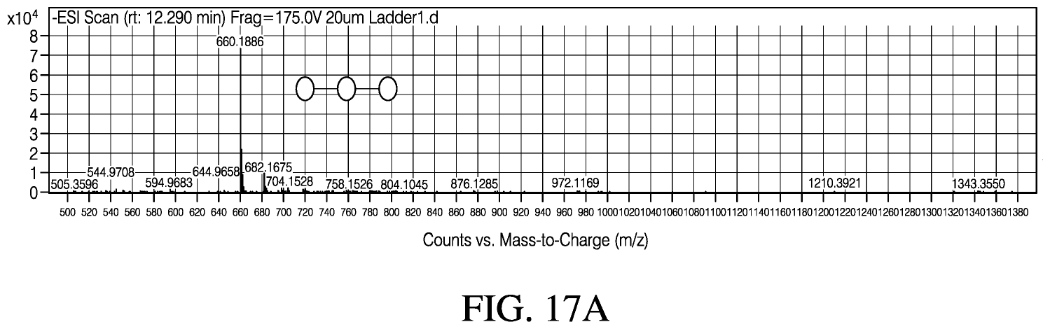

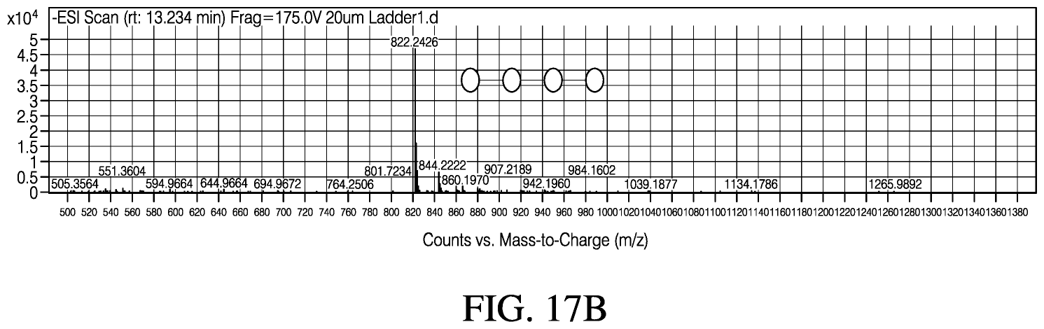

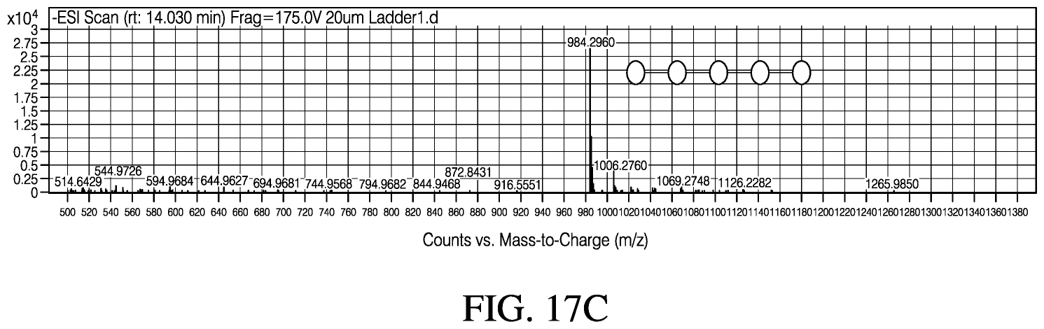

Method of Labeling Glycans with sulfanilic acid (for e.g. 3ASA): N-linked glycans were enzymatically removed with PNGase F and glycans are purified using magnetic beads. As glycans are released from a glycoprotein, it is useful to label them. N-glycans are typically labeled at their free-reducing terminus and are analyzed by methods such as high performance liquid chromatography (HPLC), UPLC/UHPLC, HILIC, capillary electrophoresis (CE), carbohydrate gel electrophoresis, fluorescence analysis, mass spectrometry (MS) and others. Glycans were identified by retention time relative to an appropriate size standard ladder. For e.g., glycans can be quantified using maltotriose (M3), maltotetrose (M4) and/or maltopentose (M5) as internal standards (see FIG. 17).

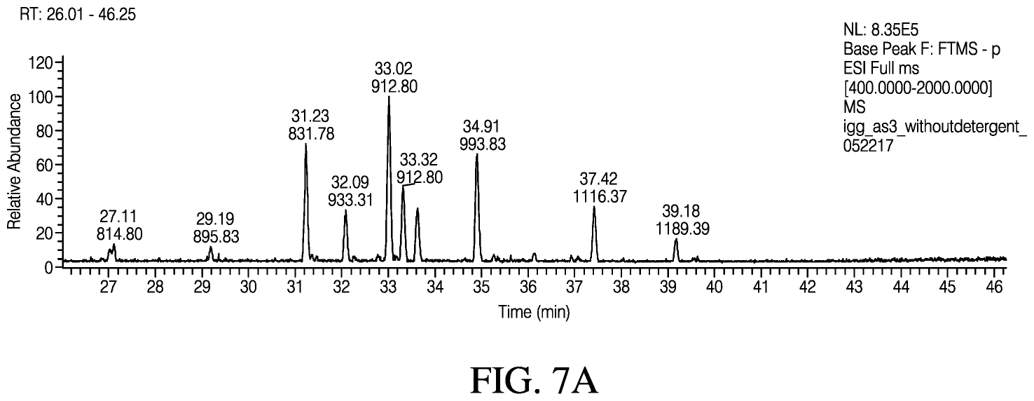

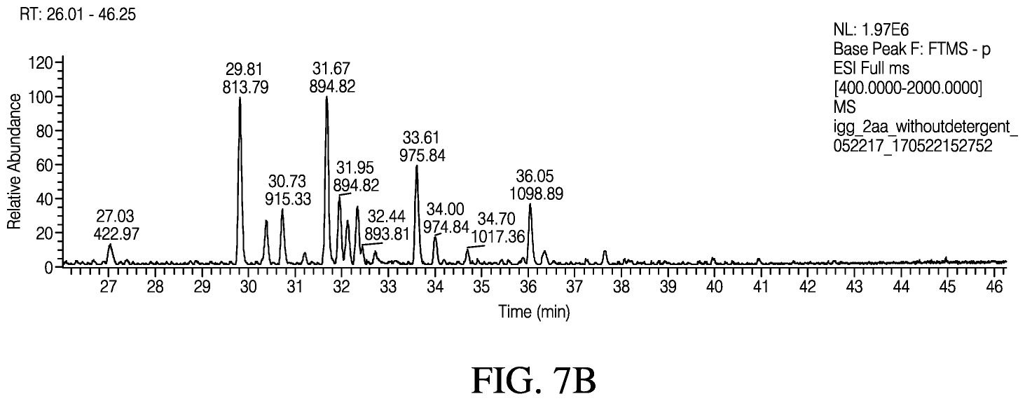

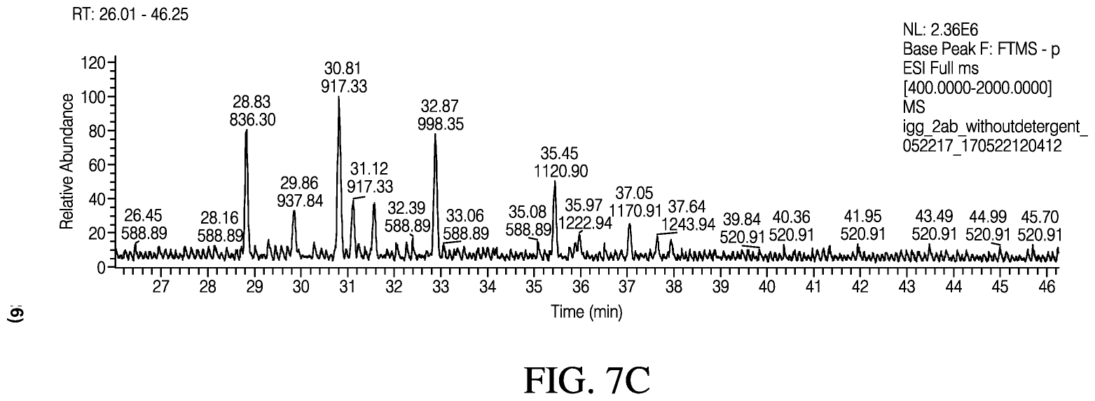

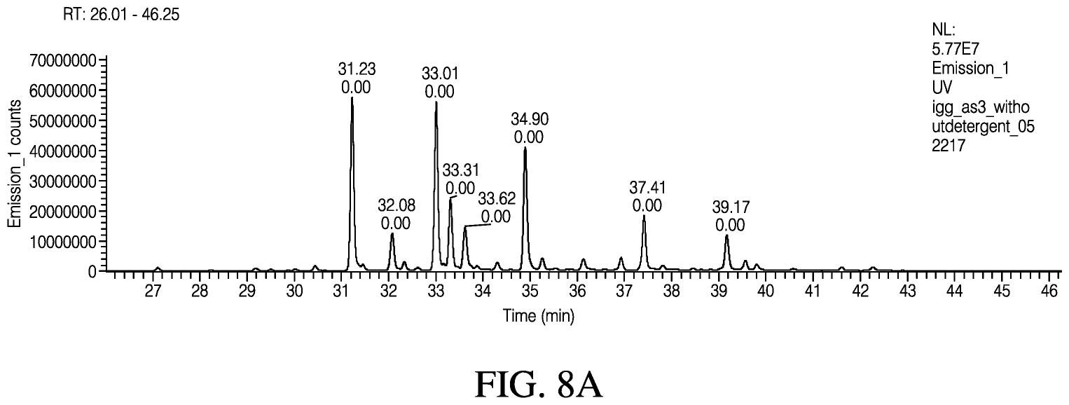

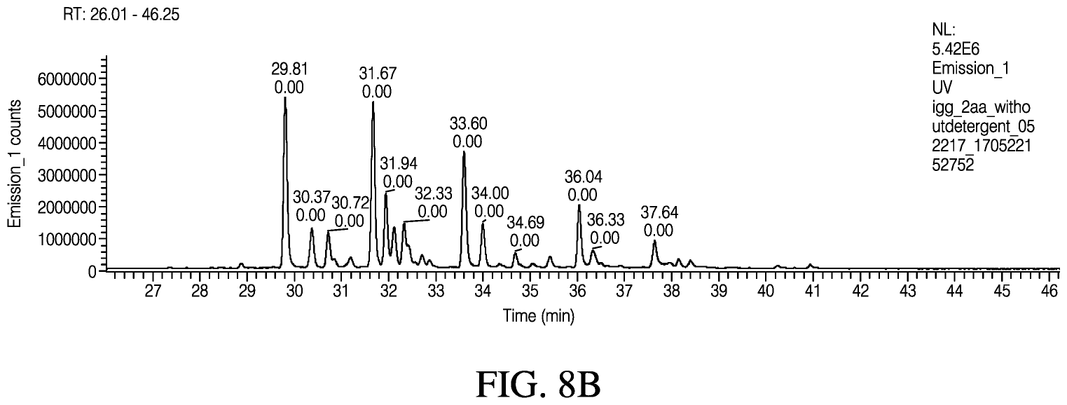

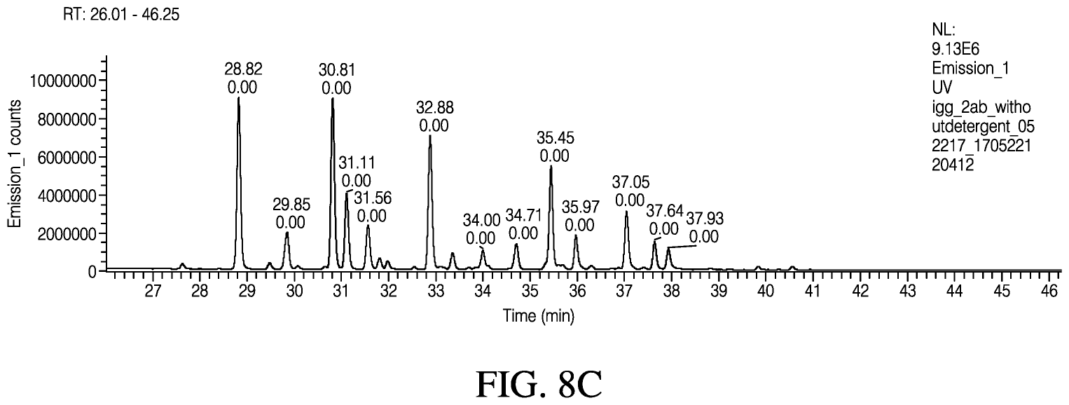

CE-MS analysis: A key feature of the CE-MS method is low sample consumption, enabling LC-MS to be performed using the same sample, thereby facilitating quantitative structural analysis using an orthogonal method for verification purposes. CE-MS experiments were performed using an Agilent 7100 capillary electrophoresis system interfaced using an Agilent G1607B orthogonal coaxial sheath sprayer to an Agilent 6520 Accurate-Mass Q-TOF mass spectrometer under the control of Mass Hunter acquisition software, version B.06.01. (Santa Clara, Calif., USA). Samples were injected hydrodynamically (100 mbar, 10 seconds) and electrophoresis separations were performed using an applied voltage of −30 kV. Reagents, sample preparation methodology and conditions used herein for CE-MS was done as described in Varadi et al., Anal. Bioanal. Chem., 2016 December; 408(30):8691-8700, hereby incorporated by reference in its entirety. FIGS. 7 and 8 show the relative abundance of release glycans from huIgG showing the comparison by using two other commercially available negative ion dyes—2AA and 2AB. In general, 3ASA (Sulfanilic acid) generated a smoother MS baseline curve (FIG. 7) and generated higher intensity fluorescence peaks compared with 2AA and 2AB.

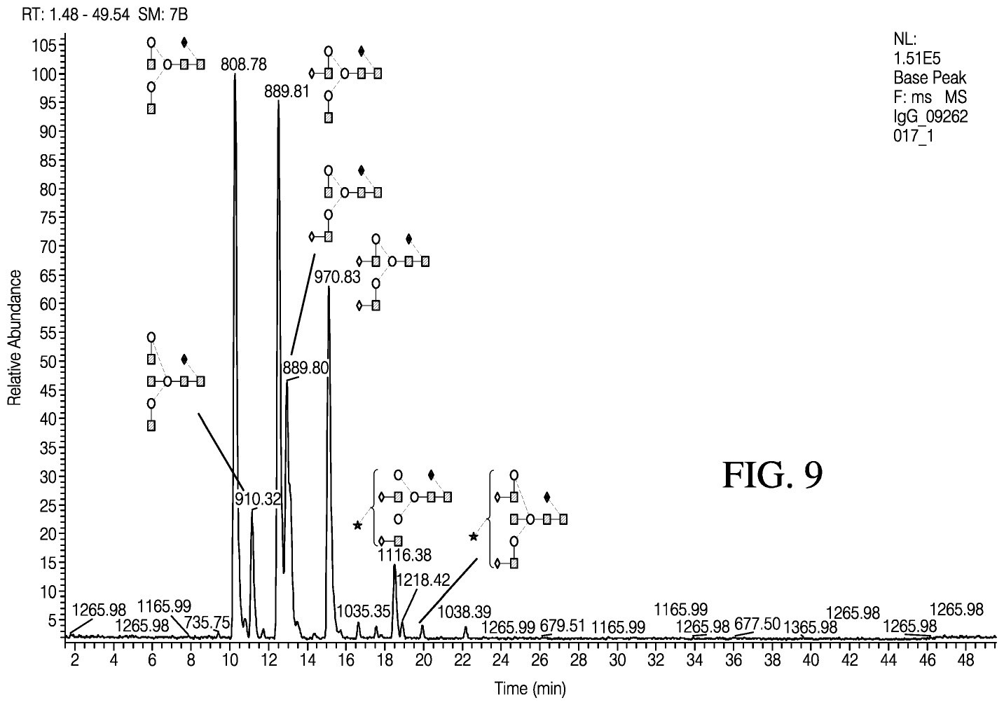

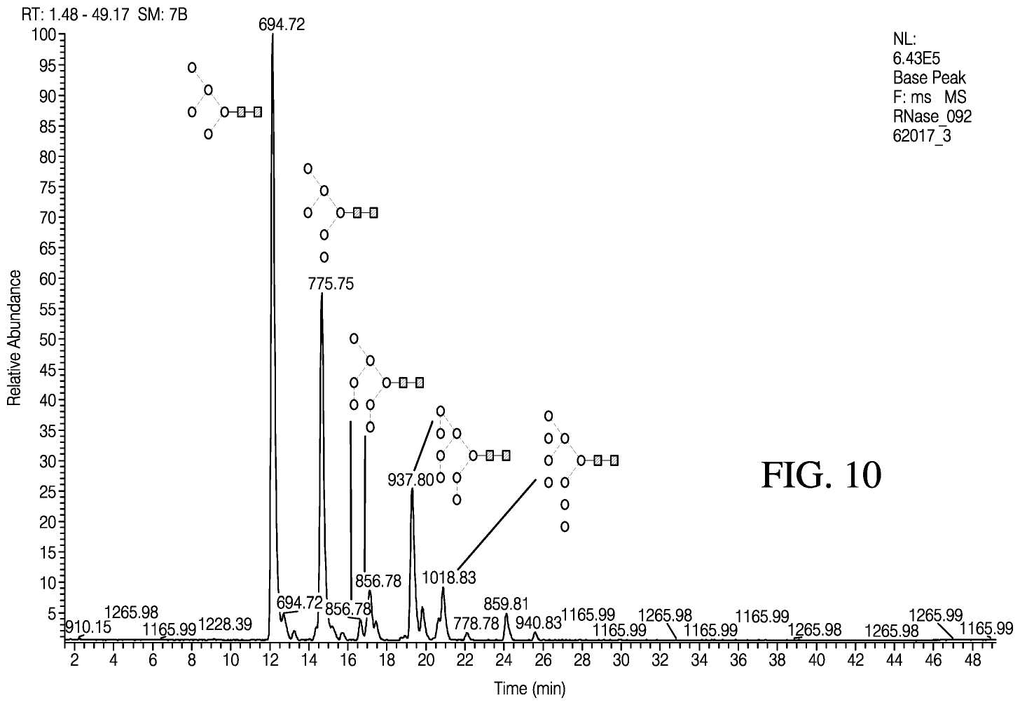

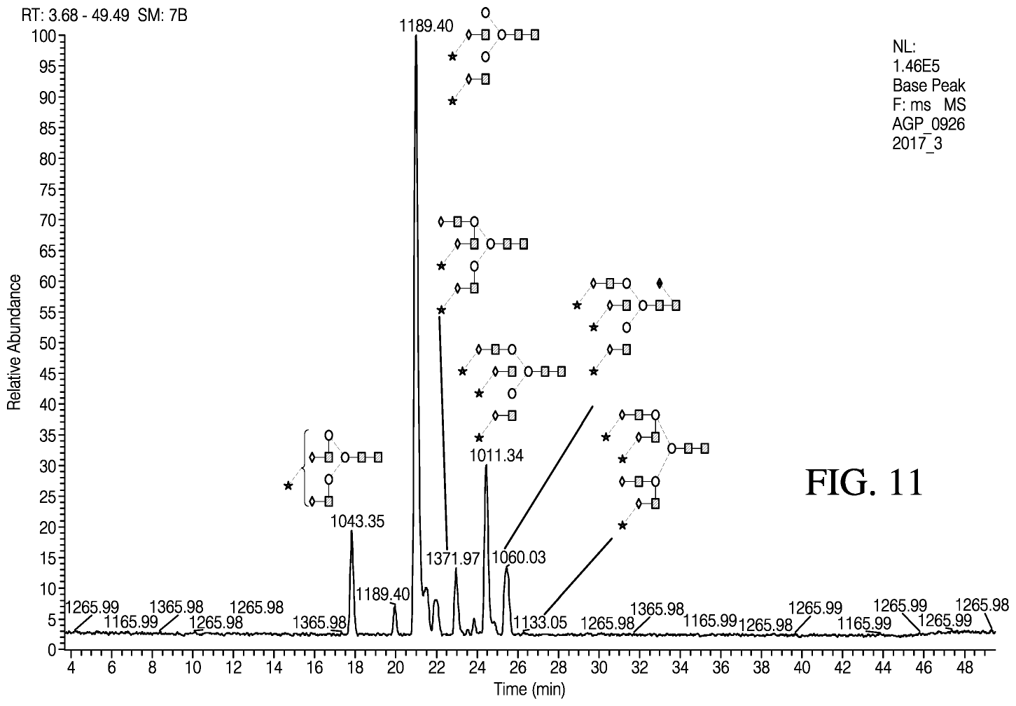

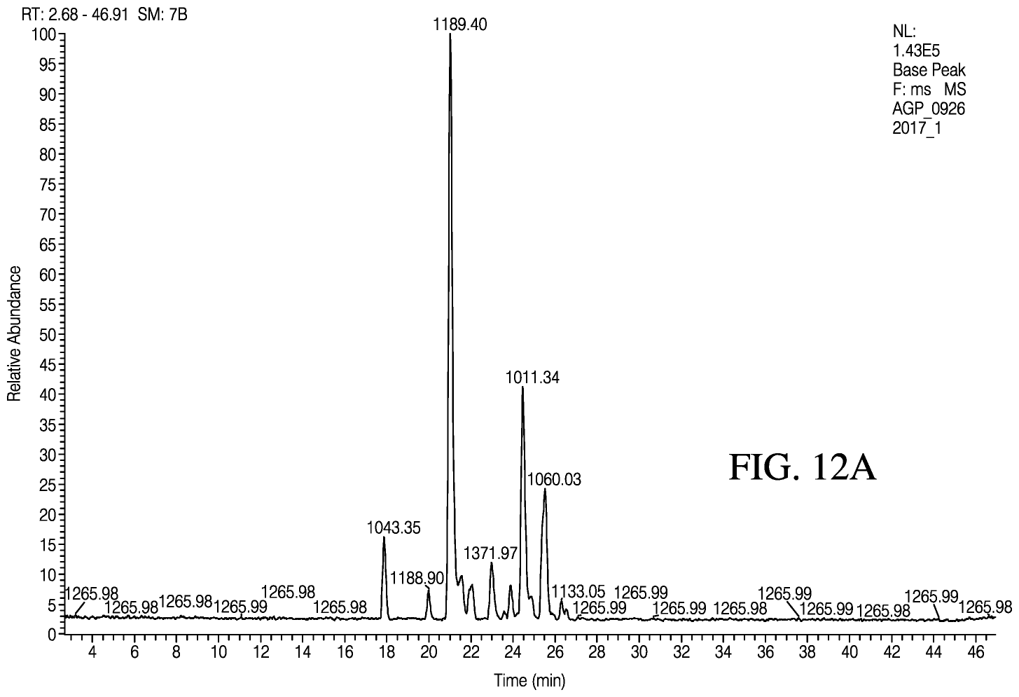

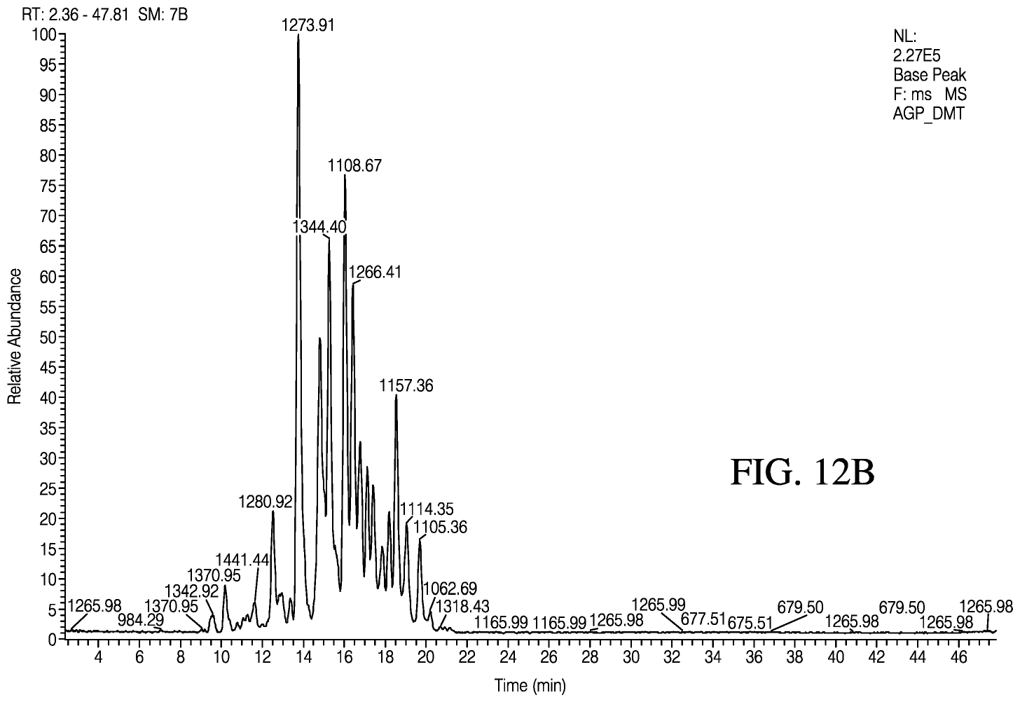

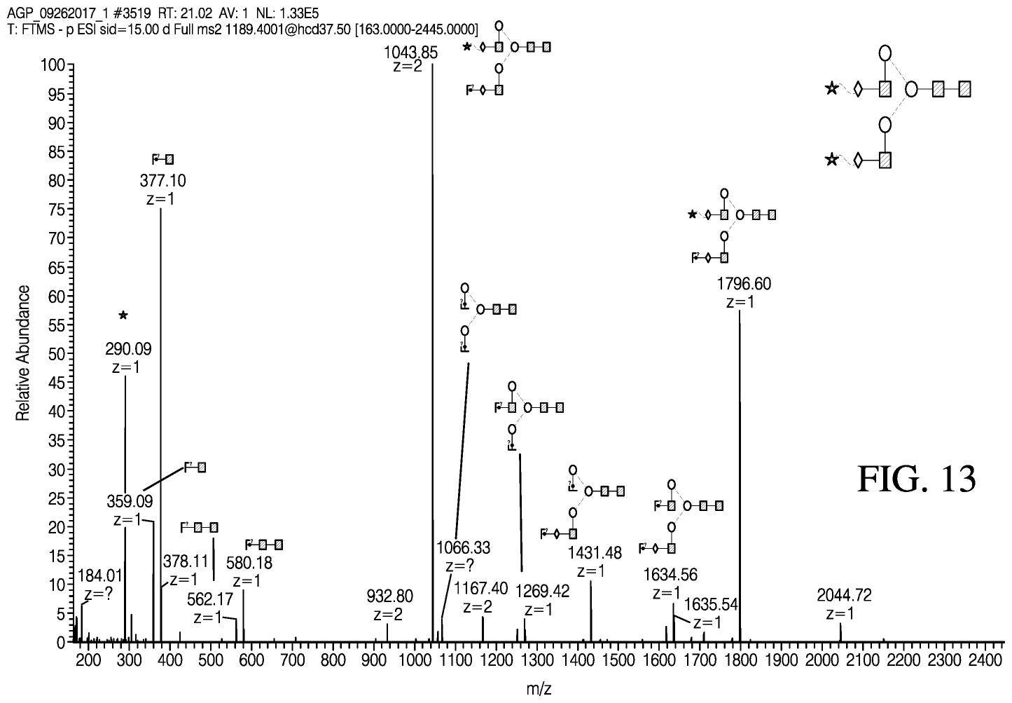

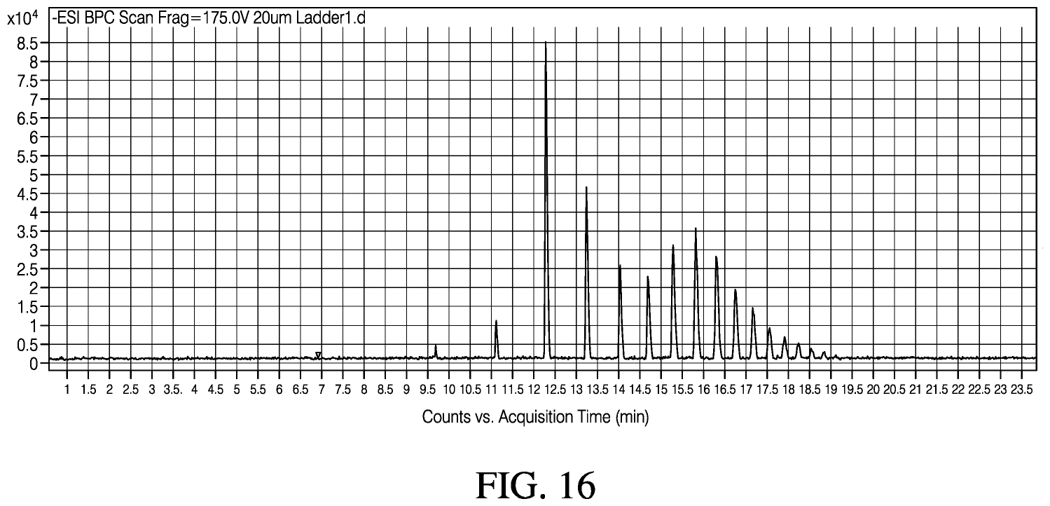

Mass spectra were collected using negative ionisation mode over the acquisition range of 500-2000 m/z with 2 GHz digitisation. FIG. 9 shows the relative abundance of N-glycans released from human IgG using mass spectrometric analysis with sulfanilic acid (3ASA). MS/MS spectra were also collected in the data dependent mode over the range of 100-2000 m/z. The ability to collect tandem mass spectral data (MS/MS) across CE peaks was determined to assist with structural annotation. As shown in FIGS. 16-17, 3ASA-labeled maltodextrin (FIG. 16) and 3ASA-labeled maltooligosaccharide ladders (FIG. 17) were used as model analytes to optimise CE-MS separation and buffer parameters. Also, FIG. 13 shows the sulfanilic acid labeled fragmentation of a highly sialylated protein AGP. FIG. 13 shows the resulting MS/MS spectrum of AGP A2 glycan which shows excellent fragmentation.

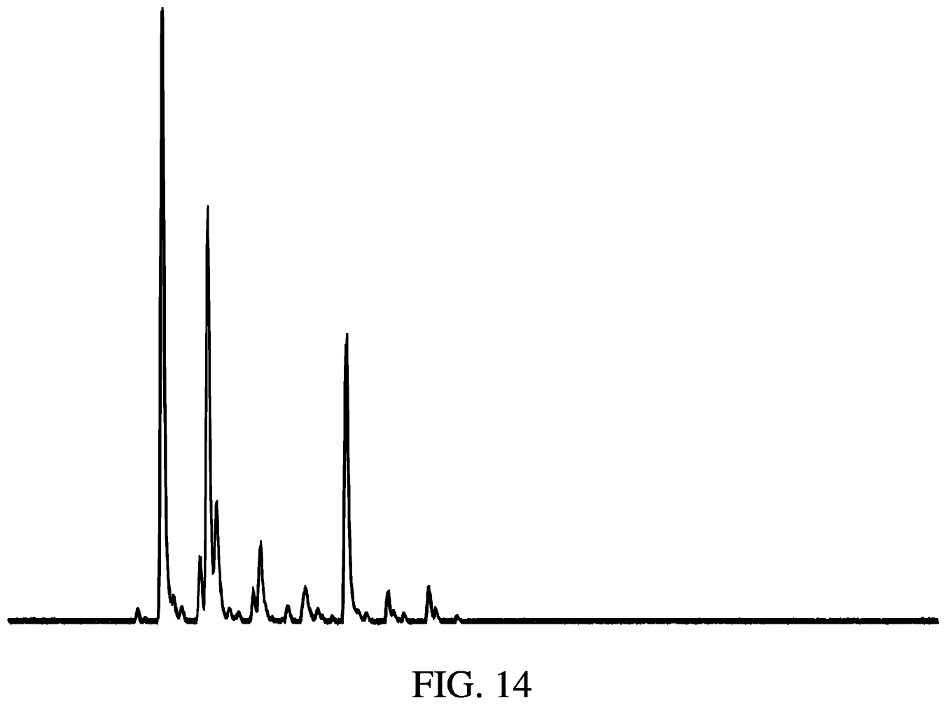

LC-MS analysis: LC-MS analysis was also performed using ¹²C 3ASA labeled N-glycan samples from the different lots of monoclonal antibody, Cetuximab. FIG. 14 shows an overlay of three base peak intensities obtained from three separate preparations of a monoclonal antibody labeled with 3ASA. N-glycans were separated using a Waters Acquity UPLC BEH Amide column (1.0×150 mm, 1.7 μm particle) connected to an Acquity UPLC equipped with online fluorescence detection (Waters Corporation, Milford, Mass., USA) under the control of MassLynx 4.1. The fluorescence excitation and emission wavelength parameters were 350 and 425 nm, respectively. Reagents, sample preparation methodology and conditions used herein for LC-MS was done as described in Varadi et al., Anal. Bioanal. Chem., 2016 December; 40$(30):8691-$700. The UPLC column outlet was coupled directly to a Waters Xevo G2 QToF mass spectrometer (Milford, Mass., USA) equipped with an electrospray ionization interface. The instrument was operated in negative ion mode with a capillary voltage of 1.80 kV under conditions as described in Varadi et al., Anal. Bioanal. Chem., 2016. Full-scan MS data was acquired over the range of 450 to 2500 m/z.

Method of Quantitative Glycan Analysis Using ¹²C and ¹³C-Sulphanilic Acid Stable Isotope Labeling

The twoplex ¹²C₆/¹³C₆-2D LC-MS platform is ideally suited for differential glycomic analysis of structurally complex glycan pools due to combination and analysis of samples in a single LC-MS injection, and due to minimal technical variation. A distinct advantage of 3ASA labeling is its permanent negative charge and negative ion polarity with strong [M-H]²⁻ pseudomolecular ions well-suited for LC-MS and CE-MS analysis of complex oligosaccharides. The stable isotope variant of sulfanilic acid—¹³C₆ isotopologue was generated which provided superior quantitative and comparative MS/MS based analyses. Quantitative glycan profiling in a twoplex manner generated extracted ion electropherograms (EIE) for ¹²C₆ ‘light’ and ¹³C₆ ‘heavy’ sulfanilic acid labeled glycan isotope clusters.

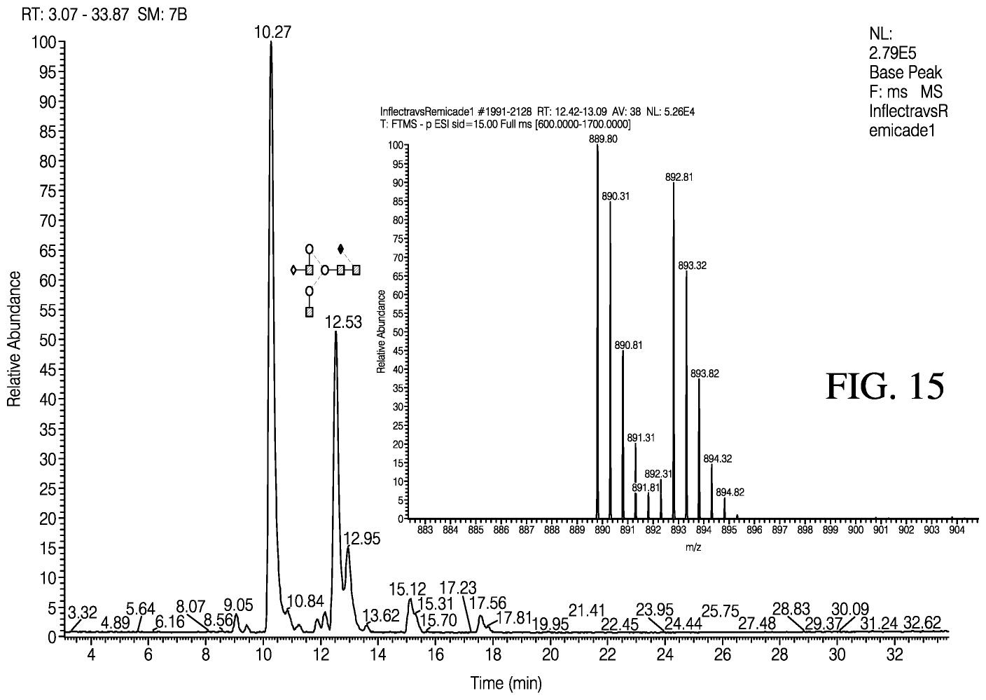

To evaluate the performance of the twoplex ¹²C₆ and ¹³C₆ 3 ASA quantitation using the developed CE-MS method, two different biosimilar drugs were labeled with ¹²C₆ and ¹³C₆ 3 ASA, cleaned and analysed by CE-MS. Quantitation was performed based on the peak area of generated EIEs for the isotope clusters of the ¹²C₆ ‘light’ and ¹³C₆ ‘heavy’ 3ASA, respectively. Resulting isotope based quantitation data is shown in FIG. 15 for the two monoclonal antibodies (INFLECTRA® and REMICADE®).

DMT-MM derivatization allows for mass-based differentiation of sialic acid linkage isomers and their simultaneous and differential glycomic analysis. DMT-MM is 4-(4,6-Dimethoxy-1,3,5-triazin-2-yl)-4-methylmorpholinium chloride. With HILIC (Hydrophilic Interaction Liquid Chromatography), separation is mediated by changes in their structural polarity. Such methods, when combined with the use of ¹³C isotopologue-labeling on the reducing terminus of glycans/saccharides, enabled the differential analysis and/or the quantitation of (¹²C/¹³C) individual glycan/saccharide isomers within complex mixtures of glycans/saccharides, such as in biological samples, medical samples, etc.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of labeling one or more glycans in a glycoprotein sample, comprising:
(d) deglycosylating the one or more glycans from the glycoprotein;
(e) contacting the one or more deglycosylated glycans of step (a) with a fluorescent reagent of Formula I: 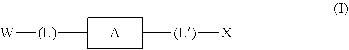
wherein,
A is an aromatic component;
X is a molecular scaffold substituent designed to react with a glycan aldehyde, ketone or an aminoglycan;
optionally attached L and L′ are linkers, that are independently (CH₂)ₙ, (OCH₂CH₂)ₘ, (CH₂)ₙ(OCH₂CH₂)ₚ or (OCH₂CH₂)ₚ(CH₂)ₙ; where n is 0 to 8, and m and p are independently 0 to 4; and,
W is a negatively charged substituent for detection;
(f) reacting the one or more deglycosylated glycans and the fluorescent reagent to form a fluorescent target product.

2. The method of labeling one or more glycans of claim 1, wherein the fluorescent target product is an labeled glycan.

3. The method of labeling one or more glycans of claims 1-2, wherein:
A is benzene, (N- or S-) substituted benzene, naphthalene, (N- or S-) substituted naphthalene, anthracene, (N- or S-) substituted anthracene, pyrene, (N- or S-) substituted pyrene, or derivatives or salts thereof;
X is an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives or salts thereof; and,
W is sulfonic acid and their derivatives or salts thereof, phosphoric acid and their derivatives or salts thereof, carboxylic acid and their derivatives or salts thereof.

4. The method of labeling one or more glycans of any of the preceding claims, wherein the activated carboxylic group is selected from: a succinimidyl, N-hydroxysuccinimidyl (NHS) ester, N-hydroxysuccinimidyl (NHS) cyanate, N-hydroxysuccinimidyl (NHS) thiocyanate, N-hydroxysuccinimidyl (NHS) isothiocyanate, and N-hydroxysuccinimidyl (NHS) isocyanate.

5. The method of labeling one or more glycans of any of the preceding claims wherein the fluorescent reagent of Formula I is a benzenesulfonic acid derivative.

6. The method of labeling one or more glycans of any of claims 1-5, where the fluorescent reagent is a compound of Formula II, III or IV: 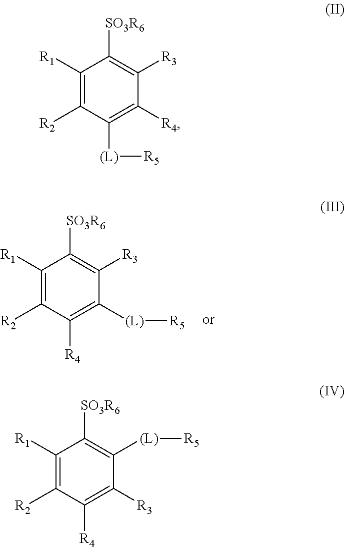
wherein,
R₁, R₂, R₃ and R₄ are independently, H, C₁-C₆-alkyl, C₁-C₆ alkoxy or halogen;
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ; where n is 0 to 8, and m and p are independently 0 to 4;
R₅ is a primary amino group, hydrazine or substituted hydrazine, hydrazide or substituted hydrazide, hydrazone or substituted hydrazone, oxime or substituted oxime, sulfonyl chloride, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds;
R₆ is H or O⁻ or OM where M is a metal ion or an ammonium ion.

7. The method of labeling one or more glycans of claim 6, wherein R₁, R₂, R₃ and R₄ are independently H, methyl, ethyl, C₃-C₆ alkyl, C₁-C₆ alkoxy or halogen.

8. The method of labeling one or more glycans of any of claims 1-5, where the fluorescent reagent is a compound of Formula V, VI or VII: 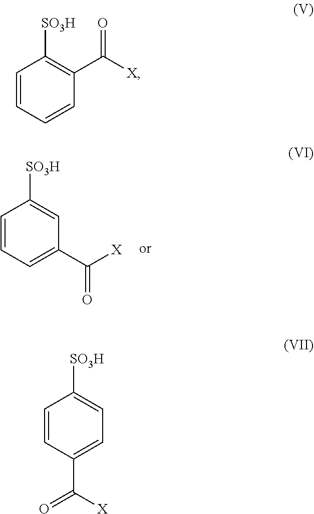
wherein, X is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof.

9. The method of labeling one or more glycans of any of claims 1-5, where the fluorescent reagent is a compound of Formula IX, X or XI: 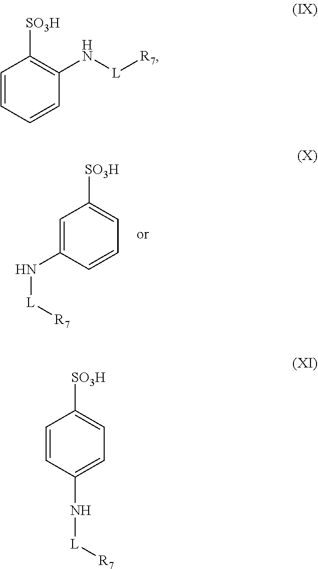
wherein,
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ where n is 0 to 8 and m and p are independently 0 to 4;
if n, m or p is 0, R₇ is H; and,
if n is 1 to 8, or if m or p are independently 1 to 4, then,
R₇ is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds.

10. The method of labeling one or more glycans of any of claims 1-5, where the fluorescent reagent is Compound 1, 2 or 3:

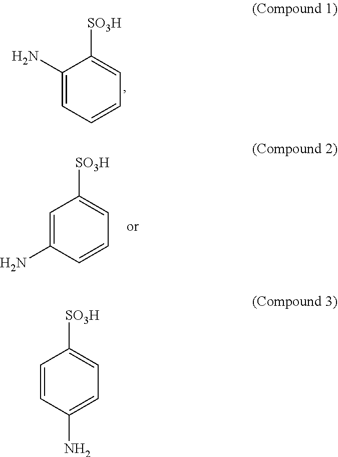

11. The method of labeling one or more glycans of any of claims 1-5, where the fluorescent reagent is Compound 9 or Compound 10: 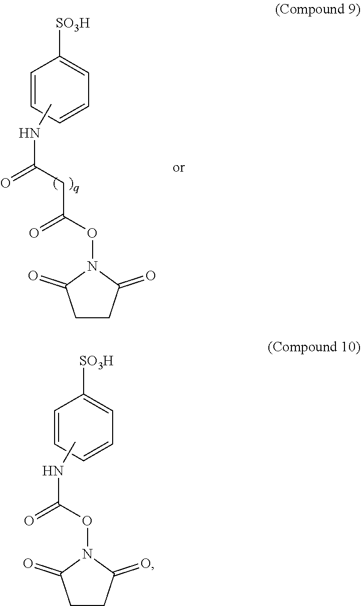
wherein, the benzenesulfonic acid is ortho-, meta- or para-substituted; and,
q is 0 to 4.

12. The method of labeling one or more glycans of any of claims 1-5 where the fluorescent reagent is Compound 11, 12 or 13: 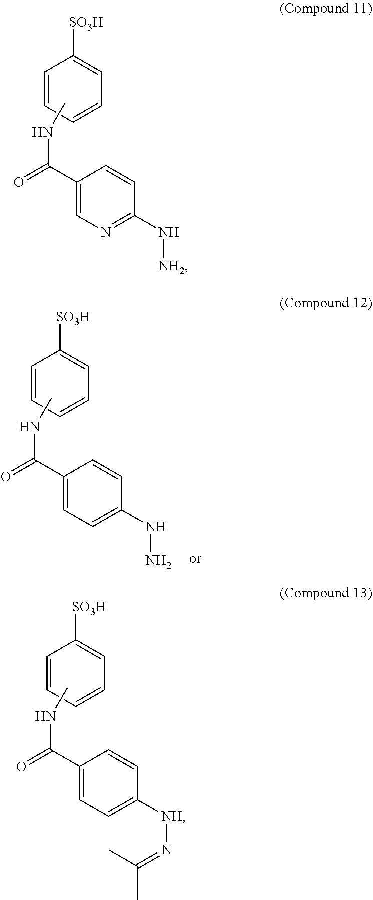
where the benzenesulfonic acid is ortho-, meta- or para-substituted.

13. The method of labeling one or more glycans of any of claims 1-5, where the fluorescent reagent is Compound 6, 7 or 8:

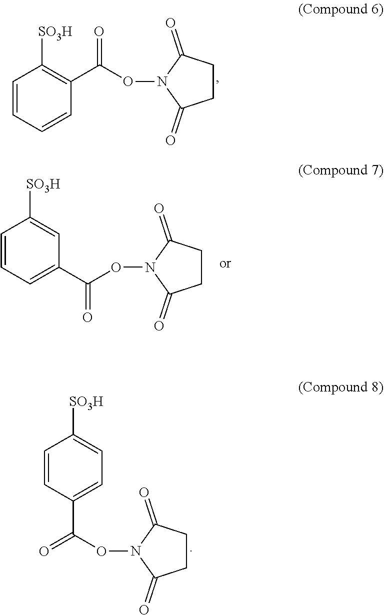 [SMILES: O=C(ON1C(=O)CCC1=O)c1ccccc1S(=O)(=O)O ; O=C(ON1C(=O)CCC1=O)c1cccc(S(=O)(=O)O)c1 ; O=C(ON1C(=O)CCC1=O)c1ccc(S(=O)(=O)O)cc1]

14. The method of labeling one or more glycans of any of the preceding claims, wherein the aromatic ring carbocycle is labeled with ¹³C, and wherein the carbocyle contains between one and seven ¹³C atoms.

15. The method of labeling one or more glycans of any of the preceding claims, wherein the glycoprotein is either in a solution or is immobilized on a support selected from the group consisting of a resin, a bead, a membrane, an array, a surface, a cartridge, a solid, a plate and a well.

16. The method of labeling one or more glycans of any of the preceding claims, wherein the deglycosylation is done either by a physical method, a chemical method or by an enzyme.

17. The method of labeling one or more glycans of any of the preceding claims, wherein the enzyme is a glycosidase.

18. The method of labeling one or more glycans of any of the preceding claims, wherein the glycosidase is PNGase F.

19. A method of detecting glycans in a sample, comprising:
(f) deglycosylating any bound glycans in the sample;
(g) contacting one or more glycans from the sample with a fluorescent reagent of Formula I: 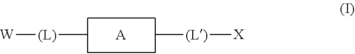
wherein,
A is an aromatic component;
X is a molecular scaffold substituent designed to react with a glycan aldehyde, ketone or an aminoglycan;
optionally attached L and L′ are linkers, that are independently (CH₂)ₙ, (OCH₂CH₂)ₘ, (CH₂)ₙ(OCH₂CH₂)ₚ or (OCH₂CH₂)ₚ(CH₂)ₙ; where n is 0 to 8, and m and p are independently 0 to 4; and,
W is a negatively charged substituent for detection;
(h) allowing the sample and the fluorescent reagent to react together to form a fluorescent target product;
(i) exciting the fluorescent target product with UV/visible light of an appropriate excitation wavelength; and,
(j) quantifying or determining the presence of the glycan in the fluorescent target product by detecting appropriate UV/visible emissions from the fluorescent target product and/or detecting the presence of the glycan in the fluorescent target product.

20. The method of detecting glycans of claim 19, wherein:
A is benzene, (N- or S-) substituted benzene, naphthalene, (N- or S-) substituted naphthalene, anthracene, (N- or S-) substituted anthracene, pyrene, (N- or S-) substituted pyrene, or derivatives or salts thereof;
X is an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives or salts thereof; and,
W is sulfonic acid and their derivatives or salts thereof, phosphoric acid and their derivatives or salts thereof, carboxylic acid and their derivatives or salts thereof.

21. The method of detecting glycans of any of claims 19-20, wherein the activated carboxylic group is selected from: a succinimidyl, N-hydroxysuccinimidyl (NHS) ester, N-hydroxysuccinimidyl (NHS) cyanate, N-hydroxysuccinimidyl (NHS) thiocyanate, N-hydroxysuccinimidyl (NHS) isothiocyanate, and N-hydroxysuccinimidyl (NHS) isocyanate.

22. The method of detecting glycans of any of claims 19-21 wherein the fluorescent reagent of Formula I is a benzenesulfonic acid derivative.

23. The method of detecting glycans of any of claims 19-22, where the fluorescent reagent is a compound of Formula II, III or IV: 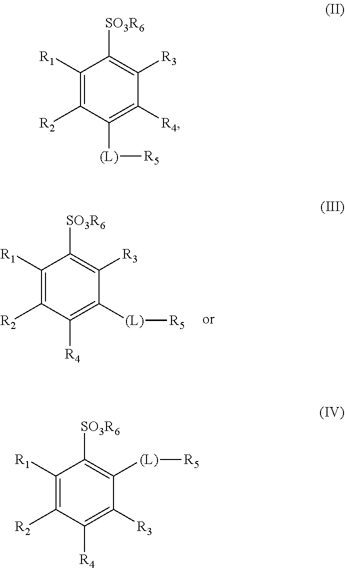
wherein,
R₁, R₂, R₃ and R₄ are independently, H, C₁-C₆-alkyl, C₁-C₆ alkoxy or halogen;
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ; where n is 0 to 8, and m and p are independently 0 to 4;
R₅ is a primary amino group, hydrazine or substituted hydrazine, hydrazide or substituted hydrazide, hydrazone or substituted hydrazone, oxime or substituted oxime, sulfonyl chloride, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds;
R₆ is H or O⁻ or OM where M is a metal ion or an ammonium ion.

24. The method of detecting glycans of any of claims 19-23, wherein R₁, R₂, R₃ and R₄ are independently H, methyl, ethyl, C₃-C₆ alkyl, C₁-C₆ alkoxy or halogen.

25. The method of detecting glycans of any of claims any of claims 19-22, where the fluorescent reagent is a compound of Formula V, VI or VII: 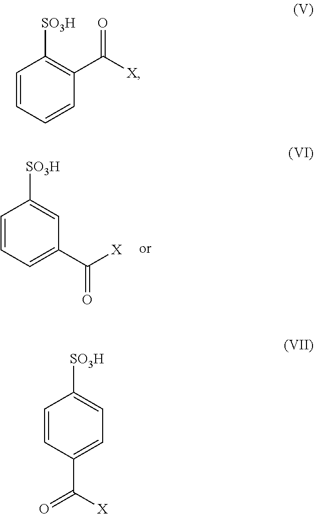
wherein, X is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof.

26. The method of detecting glycans of any of claims any of claims 19-22, where the fluorescent reagent is a compound of Formula IX, X or XI: 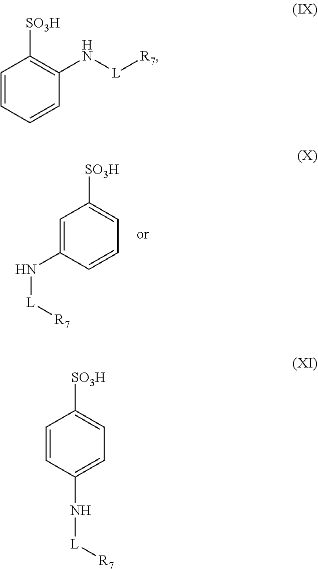
wherein,
optionally attached linker L is (CH₂)ₙ, (OCH₂CH₂)ₘ, (CH₂CH₂O)ₘ, (CH₂)ₙ(OCH₂CH₂)ₚ or (OCH₂CH₂)ₚ(CH₂)ₙ where n is 0 to 8 and m and p are independently 0 to 4;
if n, m or p is 0, R₇ is H; and,
if n is 1 to 8, or if m or p are independently 1 to 4, then,
R₇ is selected from an amine, hydrazine or substituted hydrazine, hydrazide or substituted hydrazide, hydrazone or substituted hydrazone, oxime or substituted oxime, an activated carboxylic group such as a succinimidyl or N-hydroxysuccinimidyl (NHS) ester or equivalents (including but not limited to cyanates, thiocyanate, isothiocyanates, isocyanates), carbamates, or derivatives and salts thereof of any of the preceding compounds.

27. The method of detecting glycans of any of claims any of claims 19-22, where the fluorescent reagent is Compound 1, 2 or 3:

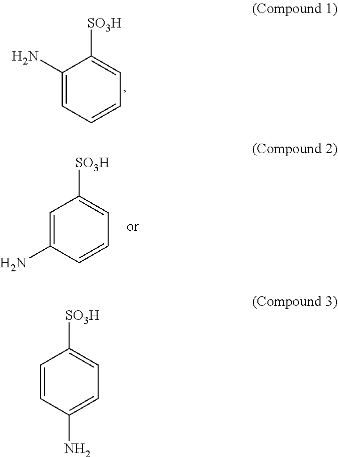

28. The method of detecting glycans of any of claims 19-22, where the fluorescent reagent is Compound 9 or Compound 10: 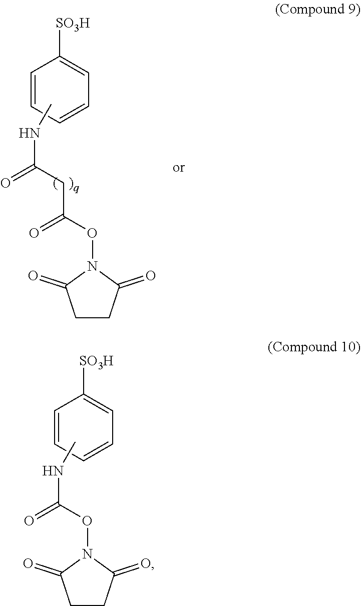
wherein, the benzenesulfonic acid is ortho-, meta- or para-substituted; and,
q is 0 to 4.

29. The method of detecting glycans of any of claims 19-22 where the fluorescent reagent is Compound 11, 12 or 13: 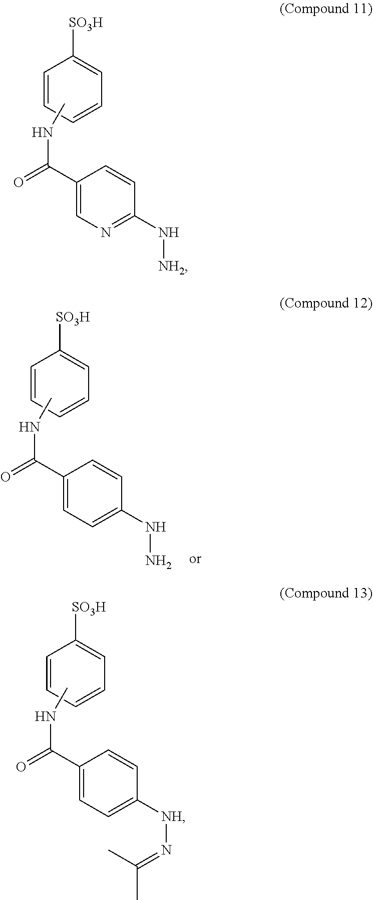
where the benzenesulfonic acid is ortho-, meta- or para-substituted.

30. The method of detecting glycans of any of claims 19-22, where the fluorescent reagent is Compound 6, 7 or 8:

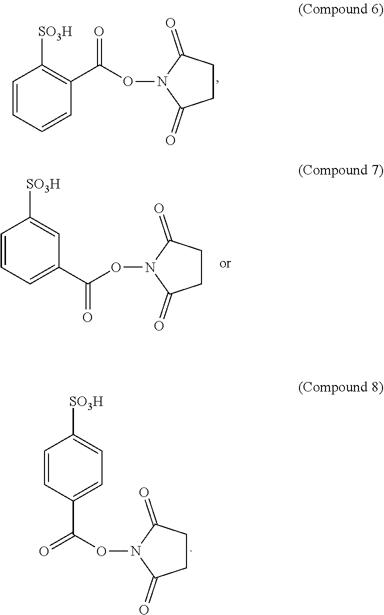 [SMILES: O=C(ON1C(=O)CCC1=O)c1ccccc1S(=O)(=O)O ; O=C(ON1C(=O)CCC1=O)c1cccc(S(=O)(=O)O)c1 ; O=C(ON1C(=O)CCC1=O)c1ccc(S(=O)(=O)O)cc1]

31. The method of detecting glycans of any of claims 19-30, wherein the aromatic ring carbocycle of any one of the compounds in claims 19-30 is labeled with either ¹²C or ¹³C, and contains between one and seven ¹²C/¹³C atoms respectively, thereby generating ¹²C/¹³C labeled isotopalogues.

32. The method of detecting glycans of claim 31, wherein the ¹²C/¹³C labeled isotopalogues are used to label glycans, and resultant ¹²C/¹³C labeled glycans are used for detection.

33. The method of detecting glycans of any of claims 19-32, wherein the detecting step is determining the mass-based differences in sialic acid linkage isomers after DMT-MM derivatization.

34. The method of detecting glycans of any of claims 19-33, wherein the detection is performed by MS (mass spectrometry), LC-MS, CE-MS, MS-MS, HPLC, HILIC, UPLC/UHPLC, UPLC/UHPLC-CE.

35. A method of labeling a glycan of claim 1, where the fluorescent reagent is a compound selected from:

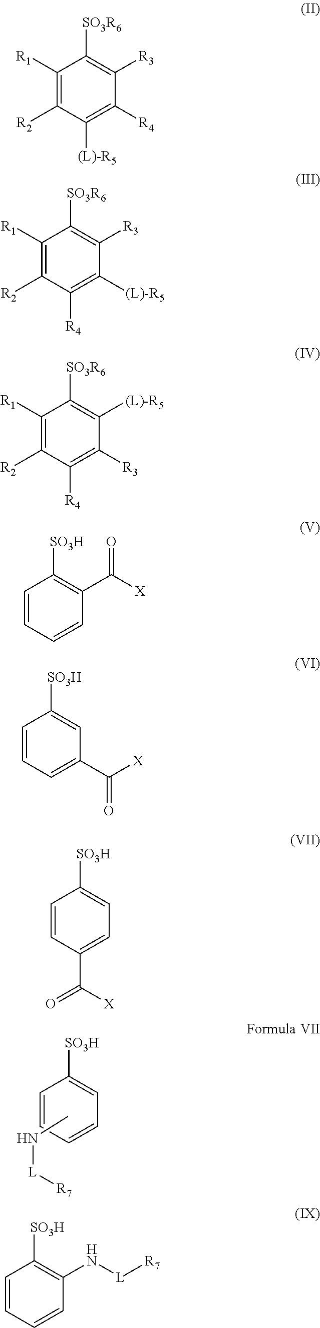

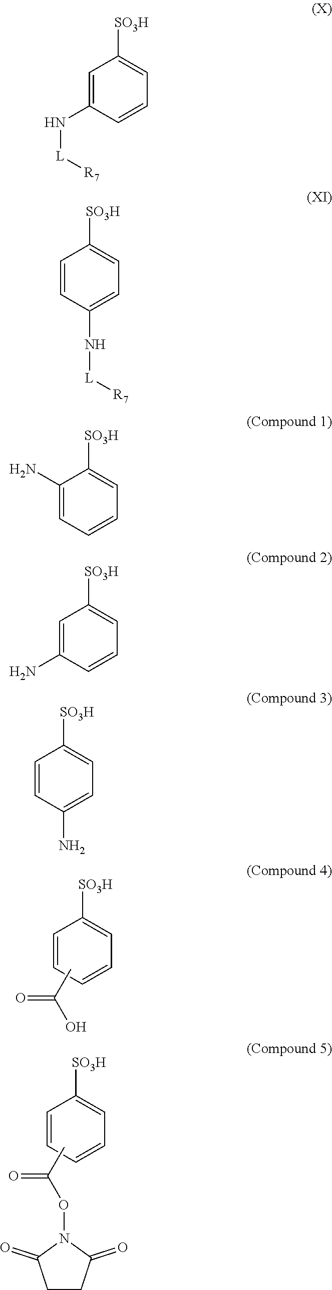

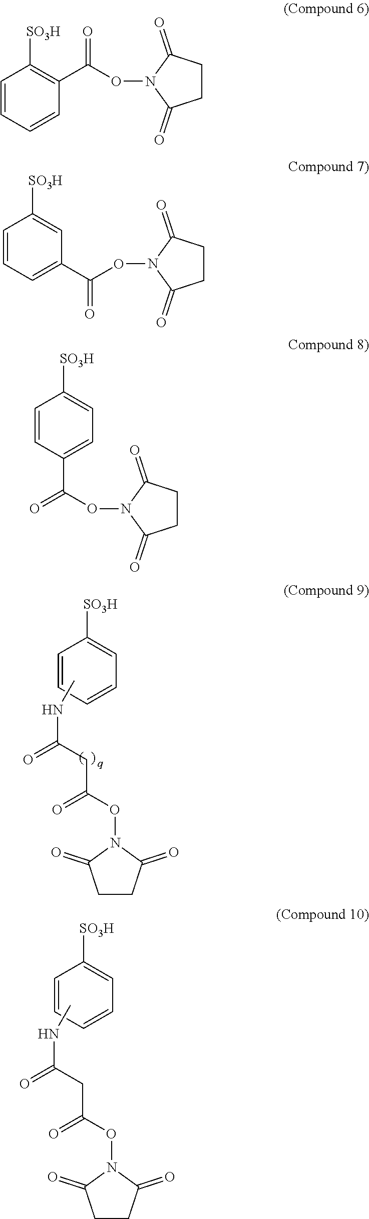

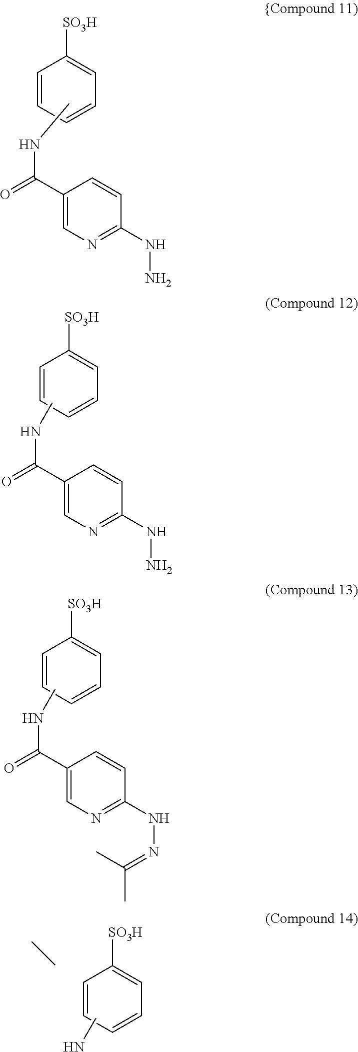

36. A method of relatively quantitating glycans in at least two samples, comprising:
a) generating ¹²C/¹³C labeled isotope analogues of any one of the fluorescent compounds of claim 35, or any one of the fluorescent compounds described in the methods claims 1-13, or the method claims 19-31 thereby resulting in a ¹²C labeled isotopologue and a ¹³C labeled isotopalogue of the selected compound;
b) generating released glycans using a glycosidase enzyme, in the first glycan sample and the second glycan sample;
c) labeling the first glycan sample with the ¹²C labeled isotopalogue of step (a) to generate ¹²C labeled glycans, and labeling the second glycan sample with the ¹³C labeled isotopalogue of step (a) to generate ¹³C labeled glycans;
d) mixing and coinjecting the ¹²C labeled and ¹³C labeled glycans into liquid chromatography or CE, and, optionally, removing the excess fluorescent labels;
e) quantitating the glycans by fluorescence; and,
f) performing tandem mass spectrometry to quantitate the ¹²C/¹³C labeled glycans by mass-based differences.

37. The method of quantitating glycans of claim 36, further comprising:
g) determining the mass-based differences in sialic acid linkage isomers after DMT-MM derivatization.

38. The method of quantitating glycans of any of claims 36-37 wherein, the glycans are N-glycans or O-glycans.

39. The method of quantitating glycans of any of claims 36-38 wherein the liquid chromatography is done by HILIC, UPLC/UHPLC or HPLC.

40. The method of quantitating glycans of any of claims 36-39, wherein the mass spectrometry detection is done on a quadrupole time of flight instrument (UPLC-FLR-QToF-MS/MS).

41. The method of quantitating glycans of any of claims 36-40 wherein, first glycan sample is an innovator biomolecule and the second glycan sample is a biosimilar biomolecule.

42. The method of quantitating glycans of any of claims 36-41 wherein, the first glycan sample is a F_{c} portion of an antibody and the second glycan sample is a F_{ab} portion of the same antibody.

43. The method of quantitating glycans of any of claims 36-42 wherein, the first glycan sample is obtained after a first treatment, and the second glycan sample is obtained after a second treatment.

44. The method of quantitating glycans of any of claims 36-43 wherein, the first glycan sample is obtained before a treatment, and the second glycan sample is obtained after the treatment.

45. The method of quantitating glycans of any of claims 36-44 wherein the first and second glycans are samples from bioreactor runs.

46. The method of quantitating glycans in a sample of any of claims 36-44, wherein the first and second glycans are disease samples.

47. The method of quantitating glycans in a sample of any of claims 36-46 wherein, the samples are antibodies.

48. A kit for detecting an analyte in a sample, wherein the kit comprises:
a) a compound selected from any one of the fluorescent compounds of claim 35, or any one of the fluorescent compounds described in the methods claims 1-13, or the method claims 19-31;
(b) instructions for covalently labeling the selected compound of a) to the analyte.

49. The kit for detecting an analyte of claim 48, wherein the analyte is a glycoprotein, a biological sample comprising a glycoprotein or part of a glycoprotein, a fusion protein or a fragment thereof, a biosimilar or a fragment thereof, an antibody or a fragment thereof, a cell or part of a cell, a virus or part of a viral protein, protein therapeutic or a fragment thereof.

50. The kit for detecting an analyte of claims 48-49, further comprising one or more of the following: a buffering agent, a purification medium, a vial comprising the analyte, an organic solvent, one or more reagents for releasing the glycan from a biomolecule, or optionally, one or more reagents to purify the released glycan from the reaction mixture.

51. The kit for detecting an analyte of any of claims 48-50, wherein the reagent for releasing the glycan from a biomolecule is selected from a physical method, a chemical or an enzyme.

52. The kit for detecting an analyte of any of claims 48-51, wherein the enzyme is a glycosidase.

53. The kit for detecting an analyte of any of claims 48-52, wherein the glycosidase is PNGase F.

54. The kit for detecting an analyte of any of claims 48-53, wherein the instructions for detecting the analyte according to the method of any one of claims.

55. The kit for detecting an analyte of any of claims 48-54, wherein the purification medium is selected from the group consisting of a resin, a bead, a membrane, an array, a surface, a cartridge, a solid support, a plate and a well.

56. The kit for detecting an analyte of any of claims 48-55, wherein the bead is a magnetic bead.

57. The kit for detecting an analyte of any of claims 48-56, further comprising instructions for labeling glycans in a sample in preparation of glycan analysis, the method comprising:
(i) treating the sample with a release reagent, such as PNGase F enzyme, with an appropriate buffer under conditions suitable for the release of the glycan from the biomolecule, thereby forming a reaction mixture;
(ii) adding beads and buffer to the reaction mixture;
(iii) separating the supernatant from the beads;
(iv) washing the beads with wash buffer;
(v) eluting the glycans from the beads with elution buffer;
(vi) performing labeling of the glycans using one or more compounds according to claim 35, thereby forming a glycan-containing solution;
(vii) optionally, removing excess compound using fresh beads; washing beads, separating the beads from excess compound/label/wash solution; and eluting glycans from the beads; and
(viii) collecting the glycan-containing solution.
